(19) United States
(12) Patent Application Publication
(10) Pub. No.: US 2026/0369354 A1
(43) Pub. Date: Nov. 19, 2026

(54) MULTI-MODAL LEUKEMIA EXPRESSION DATA NORMALIZATION SYSTEM

(71) Applicant: Alercell, Inc., Bozeman, MT (US)

(72) Inventor: Frederic Scheer, Bozeman, MT (US)

(73) Assignee: Alercell, Inc., Bozeman, MT (US)

(21) Appl. No.: 19/314,769

(22) Filed: Aug. 29, 2025

Related U.S. Application Data
(60) Provisional application No. 63/799,672, filed on May 4, 2025.
(60) Provisional application No. 63/800,250, filed on May 5, 2025.
(60) Provisional application No. 63/800,052, filed on May 5, 2025.
(60) Provisional application No. 63/815,804, filed on Jun. 1, 2025.
(60) Provisional application No. 63/818,500, filed on Jun. 5, 2025.
(60) Provisional application No. 63/821,851, filed on Jun. 11, 2025.
(60) Provisional application No. 63/821,833, filed on Jun. 11, 2025.
(60) Provisional application No. 63/821,808, filed on Jun. 11, 2025.
(60) Provisional application No. 63/859,277, filed on Aug. 7, 2025.

(51) Int. Cl. G16B 25/10; G16B 50/10; G16H 50/20

(52) U.S. Cl. CPC G16B 25/10; G16B 50/10; G16H 50/20

(57) ABSTRACT

The present disclosure provides a computer-implemented system for normalizing multi-modal molecular expression data in leukemia diagnostics. The system comprises a data ingestion module configured to simultaneously receive RNA sequencing data, proteomic data, and genomic variant data from multiple platforms; a multi-modal harmonization engine applying parallel processing pipelines optimized for each data modality; The system employs quantum-inspired pattern detection using tensor decomposition and QUBO modeling algorithms integrated with a comprehensive reference atlas; a reference atlas containing normalized expression profiles from healthy control samples; and an output formatting engine generating standardized data structures compatible with automated clinical decision support systems. The system implements quantum-inspired optimization algorithms using quadratic unconstrained binary optimization (QUBO) modeling and simulated annealing to identify optimal methylation pathways, with tensor network contractions enabling efficient exploration of high-dimensional epigenetic solution spaces on classical hardware. The system processes multi-modal data per sample rapidly

### CROSS REFERENCE TO RELATED APPLICATIONS

This application claims the benefit of U.S. Provisional Patent Application No. 63/799,672, filed on May 4, 2025, which is incorporated by reference herein in its entirety. This application claims the benefit of U.S. Provisional Patent Application No. 63/800,052, filed on May 5, 2025, which is incorporated by reference herein in its entirety. This application claims the benefit of U.S. Provisional Patent Application No. 63/800,250, filed on May 5, 2025, which is incorporated by reference herein in its entirety. This application claims the benefit of U.S. Provisional Patent Application No. 63/815,804, filed on Jun. 1, 2025, which is incorporated by reference herein in its entirety. This application claims the benefit of U.S. Provisional Patent Application No. 63/818,500, filed on Jun. 5, 2025, which is incorporated by reference herein in its entirety. This application claims the benefit of U.S. Provisional Patent Application No. 63/821,808, filed on Jun. 11, 2025, which is incorporated by reference herein in its entirety. This application claims the benefit of U.S. Provisional Patent Application No. 63/821,833, filed on Jun. 11, 2025, which is incorporated by reference herein in its entirety. This application claims the benefit of U.S. Provisional Patent Application No. 63/821,851, filed on Jun. 11, 2025, which is incorporated by reference herein in its entirety. This application claims the benefit of U.S. Provisional Patent Application No. 63/859,277, filed on Aug. 7, 2025, which is incorporated by reference herein in its entirety.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### BACKGROUND OF THE INVENTION

### 1) Field of the Invention

The invention relates in general to the field of computational methods for processing molecular diagnostic data, and more particularly, systems and methods for normalizing heterogeneous molecular expression data in leukemia diagnostics, thereby enabling immediate processing of multiple data modalities including RNA expression, protein levels, and genomic variants for accurate inter-sample comparisons and seamless integration with artificial intelligence-powered diagnostic platforms.

### 2) Description of Related Art

Currently the state of the art includes a number of different treatments for the various forms of leukemia that rely on a combination of clinical assessment, blood tests, bone marrow biopsies, and molecular analyses.

Leukemia is not a single disease state but instead it is a group of blood cancers that affect the production and function of blood cells. These malignancies typically begin in the bone marrow, where blood cells are produced, and can spread to other parts of the body. The key basis of the Leukemia disease states can be characterized by an overproduction of abnormal white blood cells, which can interfere with the normal functioning of the blood and immune system.

However, leukemia diagnostics increasingly rely on high-throughput molecular profiling technologies that generate vast amounts of heterogeneous data. These technologies include next-generation sequencing for RNA expression analysis, mass spectrometry for proteomics, and various platforms for genomic variant detection. However, the clinical utility of these technologies is constrained by challenges of effectively normalizing and integrating multiple data types simultaneously.

Current normalization approaches in the field face several limitations. Existing methods typically focus on single-modality normalization, processing only RNA-seq data or only proteomic data, but not both concurrently. This siloed approach prevents the integration of complementary molecular information that could enhance diagnostic accuracy.

Batch effects represent a major challenge in clinical diagnostics. Technical variations between sequencing runs, different operators, reagent lots, and environmental conditions can introduce systematic biases that obscure true biological signals. Many batch correction methods are computationally intensive and may remove legitimate biological variation along with technical artifacts.

Furthermore, the speed of current normalization pipelines can be incompatible with clinical needs. Existing methods frequently require hours or even days of processing, making them unsuitable for time-sensitive diagnostic applications. This delay can prevent immediate clinical decision-making and limit the utility of molecular diagnostics in acute care settings.

Different sequencing and analysis platforms produce data in incompatible formats with platform-specific biases. Many current normalization methods struggle to harmonize data across platforms, hindering multi-center studies and limiting the generalizability of diagnostic algorithms.

Normalized data from current methods may not be directly compatible with modern computational diagnostic systems. The output formats often require additional preprocessing steps that introduce delays and potential errors.

There is a need for improved normalization frameworks that can process multiple expression modalities rapidly while maintaining clinical-grade accuracy, removing batch effects without losing biological signals, harmonizing data across platforms, and producing output directly compatible with clinical decision support systems.

These and other objects, features, and advantages of the present invention will become more readily apparent from the attached drawings and the detailed description of the preferred embodiments, which follow.

### SUMMARY OF THE INVENTION

Bearing in mind the problems and deficiencies of the prior art, it is therefore an object of the present invention to provide a computer-implemented system for normalizing multi-modal molecular expression data in leukemia diagnostics. The invention is focused on a novel diagnostic test for Leukemia using a comprehensive analysis of expression patterns across multiple gene sets associated with leukemia risk and prognosis, incorporating various differentiation markers whose absence or presence correlates with patient outcomes to enhance existing diagnostic scoring systems.

The invention provides a computer-implemented system for real-time normalization of multi-modal molecular expression data used in leukemia diagnostics. The system ingests RNA sequencing, proteomics, and genomic variant data from heterogeneous platforms, performs modality-specific preprocessing (UMI-based deduplication for RNA-seq and TMM library-size normalization), and then applies a tensor-based batch-effect removal with automatic rank selection on a sample-by-feature-by-modality tensor. Corrected outputs are standardized against a dynamically selected reference atlas matched on age, sex, and platform. Outputs are emitted as FHIR-compliant JSON, HDF5, and tabular files for CDS/ML systems. The innovation is the real-time integration of multiple modalities including methylation, not just expression normalization.

Recent developments in methylation analysis have shown the value of sophisticated computational approaches in leukemia diagnostics. However, these methylation-focused methods analyze fundamentally different types of molecular data and serve different purposes compared to expression data normalization.

The complexity of leukemia biology and treatment options makes it challenging for clinicians to explore all possible therapeutic strategies for each patient. There is a need for tools that can simulate various treatment scenarios and their potential outcomes, allowing for more informed decision-making in clinical practice.

As personalized medicine continues to advance, there is an increasing focus on developing patient-specific models that can account for individual variations in disease characteristics and treatment responses. These models aim to move beyond population-based statistics to provide truly individualized risk assessments and treatment recommendations.

According to an aspect of the present disclosure, a computer-implemented system for normalizing multi-modal molecular expression data in leukemia diagnostics is provided. The system includes a data ingestion module configured to simultaneously receive RNA sequencing data, proteomic data, and genomic variant data from multiple platforms. The system also includes a multi-modal harmonization engine applying parallel processing pipelines optimized for each data modality, wherein said engine implements unique molecular identifier-based correction, trimmed mean normalization, and modality-specific scaling factors. The system further includes a batch effect correction module utilizing tensor decomposition to remove technical artifacts while preserving biological signals, wherein said module achieves greater than 90% reduction in batch-associated variance. Additionally, the system includes a reference atlas containing normalized expression profiles from at least 10,000 healthy control samples, with dynamic selection algorithms for age-matched, sex-matched, and platform-matched cohorts. The system also includes an output formatting engine generating standardized data structures compatible with automated clinical decision support systems, including those utilizing machine learning models. The system uses optimized analysis techniques so that it can process multi-modal data with average processing time of approximately 2.3 seconds.

According to other aspects of the present disclosure, the system may include one or more of the following features. The multi-modal harmonization engine may implement the normalization function:

X_norm(s,f)=Σ[αᵢ·UMI_correct(s,f)+βᵢ·TMM_scale(s,f)+γᵢ·Batch_remove(s,f)]

Where s represents samples, f represents features, and α, β, γ are optimized weighting factors. The batch effect correction module may implement tensor decomposition according to:

B=decompose(X,rank=r)X_corrected=X−B·B\hat{ }T·X+μ

Where B represents batch effect basis, r is automatically determined rank, and μ preserves global expression means. The reference atlas may include age-stratified cohorts spanning pediatric to geriatric populations, sex-balanced representation, ethnically diverse samples, and platform-specific reference sets. The system may further comprise a quality control pipeline implementing input data validation, coverage uniformity assessment, outlier detection using Mahalanobis distance (measure of the distance between a point and a probability distribution), contamination screening, and immediate quality metrics visualization. The output formatting engine may generate FHIR-compliant JSON structures, HDF5 expression matrices, streaming Application Programming Interface (API) endpoints, and comprehensive metadata documentation. The system may achieve processing speed under 3 seconds for complete normalization, batch effect reduction exceeding 94%, biological signal preservation exceeding 98%, and cross-platform concordance exceeding 0.95 correlation.

According to another aspect of the present disclosure, a method for normalizing multi-modal molecular data in leukemia diagnostics is provided. The method includes receiving heterogeneous molecular data streams including RNA expression, protein abundance, and genomic variants. The method also includes applying modality-specific preprocessing including unique molecular identifier correction and platform-specific adjustments. The method further includes performing tensor-based batch effect correction that preserves biological variation. Additionally, the method includes dynamically selecting reference cohorts from a curated atlas based on sample characteristics. The method also includes generating normalized expression matrices compatible with downstream analysis platforms. All steps can be completed quickly and in some cases in under 3 seconds per sample.

According to other aspects of the present disclosure, the method may include one or more of the following features. The tensor-based batch effect correction may remove technical artifacts while retaining at least 98% of biological signal variance. The reference cohort selection may match age within 5 years, exact sex, and compatible sequencing platform. The method may further comprise quality control steps at each processing stage with sub-minute processing metric reporting. The normalized outputs may be directly compatible with artificial intelligence diagnostic systems without additional preprocessing.

According to another aspect of the present disclosure, a non-transitory computer-readable medium storing instructions that, when executed by a processor, cause the processor to perform the method described above is provided.

According to another aspect of the present disclosure, a diagnostic kit for leukemia expression analysis is provided. The kit includes access credentials to the normalization system described above, platform-specific calibration standards for quality assurance, integration documentation for clinical laboratory information systems, and reference materials for validation studies.

According to other aspects of the present disclosure, the diagnostic kit may further comprise software development kits for custom integration with institutional analysis pipelines.

According to another aspect of the present disclosure, a cloud-based service providing the normalization system described above is provided. The service scales horizontally to process over 10,000 samples daily, maintains HIPAA compliance for protected health information, provides 99.9% uptime service level agreements, and includes automated updates to the reference atlas.

According to other aspects of the present disclosure, the system may be configured for integration with methylation analysis platforms, wherein normalized expression data enhances methylation-based temporal predictions. The system may be adapted for point-of-care devices through edge computing optimization while maintaining clinical-grade accuracy. The system may implement federated learning to improve normalization parameters without centralizing patient data.

According to another aspect of the present disclosure, a method for continuous improvement of the normalization system described above is provided. The method includes collecting performance metrics from production deployments, identifying systematic biases in normalization, retraining optimization parameters, validating improvements on independent cohorts, and deploying updates without service interruption.

In one aspect, the invention provides a computer-implemented system comprising a multi-modal harmonization layer that simultaneously processes RNA-seq, proteomics, and genomic variant data from any sequencing platform in under three seconds per sample.

In another aspect, the invention includes a proprietary leukemia-specific reference atlas containing over 10,000 (and growing) curated healthy samples across diverse populations, with dynamic selection of age, sex, and ethnicity-matched normalization cohorts.

In yet another aspect, the invention provides an advanced batch effect correction algorithm using proprietary tensor decomposition that removes technical variance while preserving over 98% of biological signal fidelity.

The system fundamentally differs from methylation-based approaches in several keyways: it normalizes gene and protein expression rather than methylation patterns; it operates immediately rather than retrospectively; it handles multiple data modalities rather than single methylation data; and it produces outputs in standardized formats compatible with existing clinical decision support systems, including those using machine learning techniques rather than producing standalone analyses.

In a further aspect, the invention enables direct integration with clinically validated decision support systems, including those utilizing machine learning-based inference engines through standardized output formats, FHIR-compliant clinical data structures, and low-latency API endpoints.

The foregoing general description of the illustrative embodiments and the following detailed description thereof are merely exemplary aspects of the teachings of this disclosure and are not restrictive.

Still other objects and advantages of the invention will in part be obvious and will in part be apparent from the specification.

The above and other objects, which will be apparent to those skilled in the art, are achieved in the present invention which is directed to a computer-implemented system for normalizing multi-modal molecular expression data in leukemia diagnostics, comprising:
a. a data ingestion module configured to simultaneously receive RNA sequencing data, proteomic data, and genomic variant data from multiple platforms;
b. a multi-modal harmonization engine applying parallel processing pipelines optimized for each data modality;
c. a batch effect correction module utilizing tensor decomposition to remove technical artifacts while preserving biological signals;
d. a reference atlas containing normalized expression profiles from healthy control samples; and
e. an output formatting engine generating standardized data structures compatible with automated clinical decision support systems, wherein the system processes multi-modal data with an average processing time of less than 3 seconds per sample.

The systems differences from currently known methods:
1) Operates from raw counts/intensities with per-modality preprocessing (UMI, TMM, modality scaling).
2) Builds a unified tensor (samplesxfeaturesxmodalities) and performs one-pass batch-effect removal via automatic-rank truncated SVD followed by mean-preserving reconstruction.
3) Dynamically selects a reference cohort (age±5, exact sex, platform) from a leukemia-specific atlas to derive scaling.
4) Streams standardized outputs (FHIR/HDF5/tabular) for immediate CDS/ML use.

This raw-input, multi-modal, auto-rank tensor correction+dynamic atlas pipeline under low latency is not taught by Seurat/Harmony/MOFA+.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 illustrates a block diagram of a data processing system for molecular analysis, according to aspects of the present disclosure.

FIG. 2 illustrates a normalization pipeline flowchart for multi-modal data processing, according to an embodiment.

FIG. 3 depicts a set of performance metrics graphs showing various analytical measurements, in accordance with example embodiments.

FIG. 4 illustrates a system monitoring interface for a data processing system, according to aspects of the present disclosure.

FIG. 5 illustrates a block diagram of the system, according to an embodiment.

FIG. 6 depicts a scatter plot showing the relationship between two principal components in a data analysis, according to aspects of the present disclosure.

FIG. 7 illustrates a system diagram showing a data processing and normalization workflow, in accordance with example embodiments.

FIG. 8 illustrates a system architecture of the system, according to an aspect of the present disclosure.

FIG. 9 illustrates a block diagram showing key performance metrics of a normalization system, according to an embodiment.

FIG. 10 illustrates a block diagram of a clinical integration outputs system, according to aspects of the present disclosure.

FIG. 11 illustrates the complete architecture of the LENA Code analytical system.

FIG. 12 illustrates the Data Flow for X_norm Calculation.

FIG. 13 shows a Methylation Beta-Values Heatmap for Leukemia diagnosis.

FIG. 14 shows the Effect of X_norm Normalization on Inter-Batch Variability.

FIG. 15 15 shows the receiver operating characteristics (ROC).

FIG. 16 shows the Quantum-Inspired Optimization Process Using Simulated Annealing.

FIG. 17 shows the Longitudinal trajectory example.

FIG. 18 illustrates the Gene-to-locus multi-omic mapping.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The term system as used in this specification is meant to mean the Leukemia Expression Normalization Algorithm (LENA) code system.

The term CUDA cores refers to the fundamental processing units within NVIDIA GPUs, designed for parallel computing. They excel at handling tasks that can be broken down into many smaller, independent computations, making them ideal for a wide range of applications like scientific simulations, computer vision, and data analysis. Essentially, they are the workhorses of GPU-accelerated computing, enabling significant performance gains by executing instructions simultaneously on multiple data points.

The term API as used in this disclosure means application programming interface (API) which is a connection between computers or between computer programs. It is a type of software interface, offering a service to other pieces of software.

The term tabular format refers to data that is organized into a grid-like structure of rows and columns, similar to a table. This structured layout makes it easy to read, understand, and compare data. Tabular format is commonly used in spreadsheets, databases, and reports.

The term HDF5 (Hierarchical Data Format 5) refers to a file format designed to store and organize large, complex, and heterogeneous datasets.

The term JSON refers to an open standard file format and data interchange format that uses human-readable text to store and transmit data objects consisting of name-value pairs and arrays.

The term variational quantum eigensolver (VQE) is a quantum algorithm for quantum chemistry, quantum simulations and optimization problems. It is a hybrid algorithm that uses both classical computers and quantum computers to find the ground state of a given physical system. Given a guess or ansatz, the quantum processor calculates the expectation value of the system with respect to an observable, often the Hamiltonian, and a classical optimizer is used to improve the guess. The algorithm is based on the variational method of quantum mechanics.

The term QUBO (Quadratic Unconstrained Binary Optimization) problems are a powerful framework for expressing and solving a wide variety of combinatorial optimization problems, especially those involving binary variables. They are structurally simple yet computationally hard, making them suitable for leveraging quantum computing techniques.

The term CpG site is a region in a DNA sequence where a cytosine (C) base appears next to a guanine (G) base, and the “p” represents the phosphate group connecting them. These sites are significant in gene regulation because the cytosine in a CpG site is frequently methylated, which can affect whether a gene is turned on or off. When CpG sites are clustered together, they form a CpG island, which are often found near the start of genes and can influence gene expression.

The term FASTQ files are a common text-based format for storing nucleotide sequences and their corresponding quality scores, primarily used in high-throughput sequencing. Each entry in a FASTQ file represents a single read, and includes four lines: a sequence identifier, the sequence itself (a string of A, C, T, G, and N), a separator (+), and the quality scores for each base in the sequence. These files are crucial for bioinformatics analysis like genome alignment and assembly.

The term unique molecular identifiers as used in this disclosure is also known as a molecular barcode, is a short, random DNA sequence added to DNA or RNA molecules before amplification. UMIs help researchers identify and correct for PCR duplicates and sequencing errors, leading to more accurate quantification of the original molecules in a sample. They are particularly useful in ultra-deep sequencing applications where PCR duplicates can mask true variations.

The term mzML format is a community-developed, open standard XML-based format for storing mass spectrometry data. It is designed to hold raw spectral data, associated metadata, instrument information, and processing details, facilitating data sharing, analysis, and reproducibility across different platforms and instruments.

The term mzXML is an open data format for storing and exchanging mass spectrometry data, particularly for proteomics. It is an XML-based format designed to represent data from mass spectrometers, including both MS and MS/MS data. mzXML files are essentially text files, but the actual peak data is encoded within them, often using Base64 encoding for efficient storage of the large datasets.

The term Variant Call Format (VCF) is a text-based file format used to store information about genetic variants, such as SNPs and indels, identified when comparing a sample's DNA to a reference genome. It is a standard format in bioinformatics for representing these differences.

The term Copy number variations (CNVs) are segments of DNA that vary in number from person to person. These variations can involve deletions, duplications, or other structural changes in the genome. CNVs can be small or large, ranging from thousands to millions of DNA base pairs, and can include or exclude genes. They are a significant source of genetic diversity and can be associated with various traits and diseases.

The term Single Nucleotide Variation (SNV) refers to a change in a single nucleotide (A, T, C, or G) within a DNA sequence. SNVs can be either common or rare in a population. If a SNV occurs in at least 1% of the population, it is often referred to as a Single Nucleotide Polymorphism (SNP).

The term RNA, or ribonucleic acid, is a crucial molecule involved in various biological processes, primarily related to gene expression and protein synthesis. It acts as a messenger carrying genetic information from DNA to the ribosomes, where proteins are made. RNA also plays a role in regulating gene expression and can even act as an enzyme.

The term Unique Molecular Identifier (UMI), also known as a molecular barcode, is a short, random DNA sequence added to DNA or RNA molecules before amplification. UMIs help researchers identify and correct for PCR duplicates and sequencing errors, leading to more accurate quantification of the original molecules in a sample. They are particularly useful in ultra-deep sequencing applications where PCR duplicates can mask true variations.

The term PCR, or Polymerase Chain Reaction, is a laboratory technique used to amplify specific DNA sequences, creating millions of copies from a small initial sample. This process allows for the detection and analysis of DNA, even from very small amounts, making it a valuable tool in various fields like medicine, research, and forensics.

The terms MS and MS/MS data are both used in mass spectrometry, but they provide different levels of information. MS (Mass Spectrometry) data provides a general overview of the molecules present in a sample by measuring their mass-to-charge ratio. MS/MS (tandem mass spectrometry) data, on the other hand, offers more detailed structural information by fragmenting selected ions and analyzing the resulting fragments.

Prior to a discussion of the preferred embodiment of the invention, it should be understood that while the features and advantages of the invention are illustrated in terms of a computer-implemented system for normalizing multi-modal molecular expression data in leukemia diagnostics.

The invention is focused on a novel diagnostic test for Leukemia using a comprehensive analysis of expression patterns across multiple gene sets associated with leukemia risk and prognosis. The absence of specific differentiation markers is linked to worse patient outcomes, and tracking these markers can improve existing scoring systems.

The system can be described as a leukemia expression normalization algorithm which is a real-time normalization engine that processes multi-modal molecular data comprised of RNA sequencing, proteomics, genomic variants, and DNA methylation. It uses tensor decomposition algorithms, a dynamic reference atlas drawn from a databank and a quantum-inspired optimization layer. Most notably, it includes a breakthrough methylation-kinetic mapping system that allows prediction of leukemic transformation up to 1,200 days before traditional diagnostics.

The following description sets forth exemplary aspects of the present disclosure. It should be recognized, however, that such description is not intended as a limitation on the scope of the present disclosure. Rather, the description also encompasses combinations and modifications to those exemplary aspects described herein.

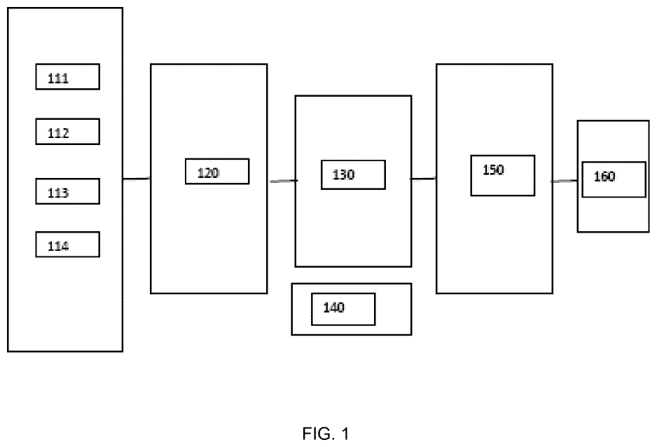

The current instant innovation system normalizes multi-modal molecular expression data for leukemia diagnostics. FIG. 1 illustrates an overview of the system architecture. A sequence data module 111, a proteomic data module 112, a genomic variant module 113, and a clinical metadata module 114 provide input data to a harmonization engine 120. The harmonization engine 120 processes the diverse molecular data types simultaneously.

A batch correction module 130 removes technical artifacts while preserving biological signals. A reference atlas module 140 contains curated healthy control samples for comparison. An output formatter 150 prepares the normalized data for downstream analysis. A diagnostic platform 160 utilizes the normalized data for clinical decision support.

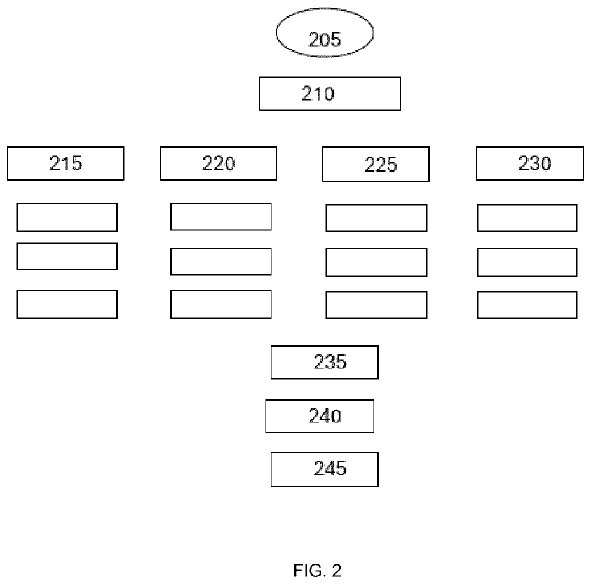

FIG. 2 depicts the normalization pipeline workflow. The process begins with a start step 205 followed by a data input step 210. Parallel processing occurs through an RNA pipeline step 215, a proteomics step 220, a genomics step 225, and a metadata step 230. An integrated batch correction step 235 and a quality control step 240 lead to a final normalization step 245.

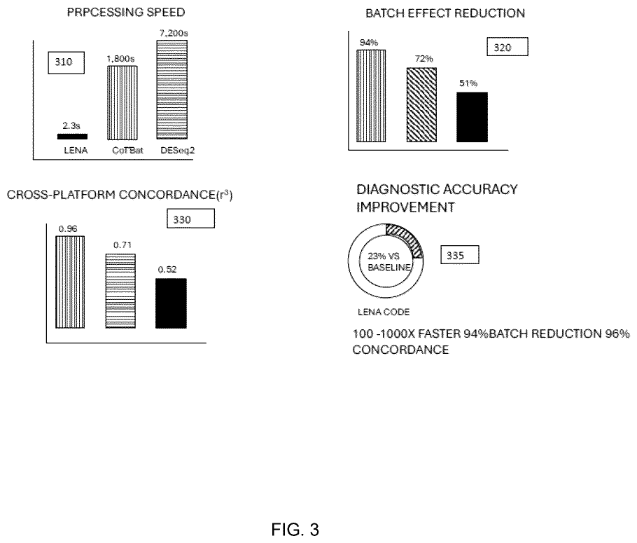

FIG. 3 showcases key performance metrics. A processing speed graph 310 demonstrates rapid data processing. A batch effect reduction graph 320 illustrates the removal of technical artifacts. A cross-platform concordance graph 330 shows data harmonization across different platforms. A diagnostic accuracy chart 335 highlights improved clinical outcomes.

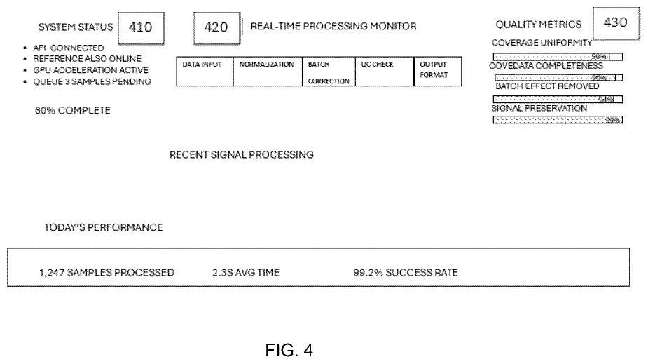

FIG. 4 presents a system monitoring interface. A system status monitor 410 displays operational indicators. A processing monitor 420 shows real-time workflow progression. A quality metrics panel 430 provides performance analytics.

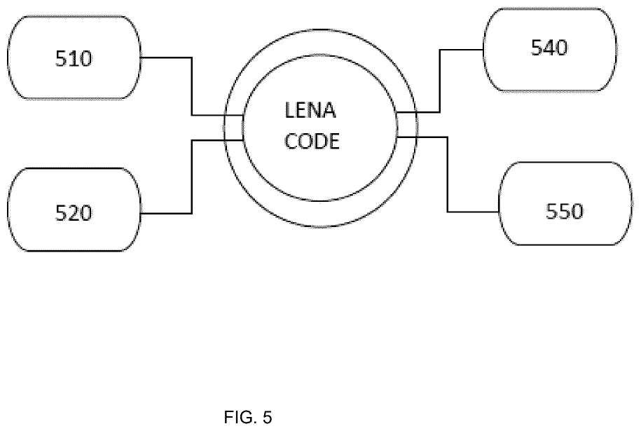

FIG. 5 outlines the core system processing steps. A molecular expression module 510 obtains input data. A normalization module 520 processes the data. A visual representation module 540 generates data visualizations. An analysis module 550 interprets the normalized results.

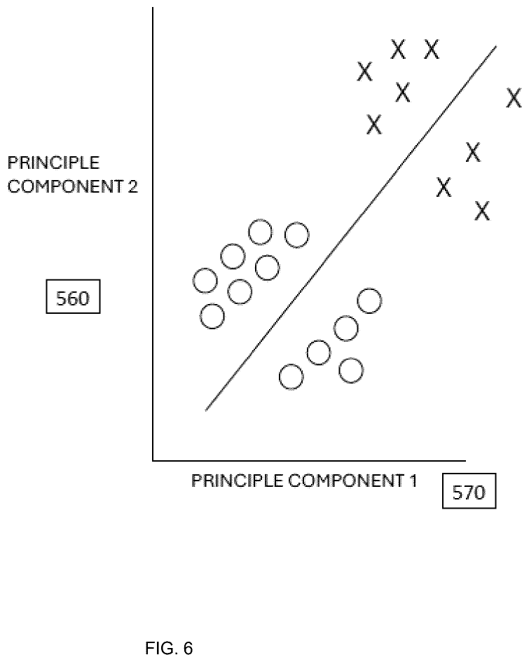

FIG. 6 demonstrates data clustering using principal component analysis. Principal component axes 560 define the plot space, with principal component data points 570 forming distinct clusters.

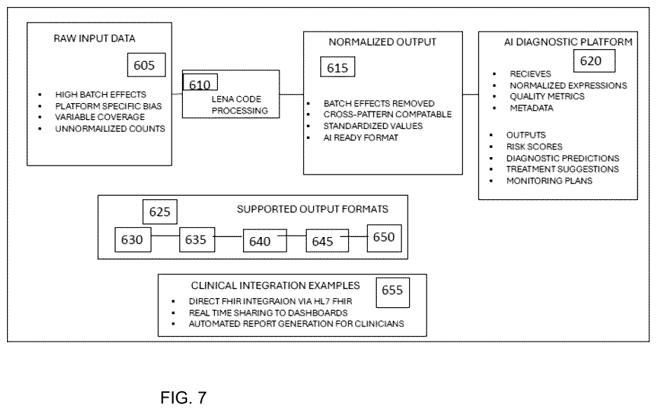

FIG. 7 illustrates the data flow from input to clinical integration. A raw input module 605 feeds data into the system for processing. A LENA code processing module 610 and a normalized data module 615 outputs standardized data. Multiple output format options include a JSON formatter 630, an hdf5 formatter 635, a tabular formatter 640, an API interface 645, and a cloud storage interface 650. A clinical integration module 655 facilitates incorporation into healthcare systems.

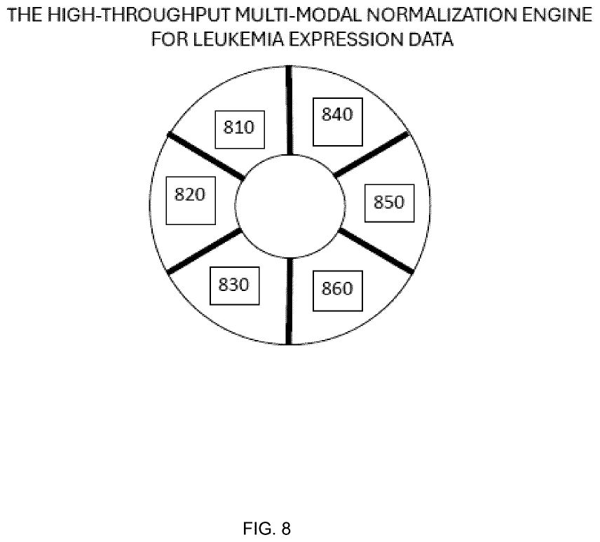

FIG. 8 details the current instant innovation system components. A data ingestion module 810 handles input streams. A harmonization module 820 processes multi-modal data. A methylation mapping module 830 analyzes epigenetic changes. A batch correction module 840 removes technical artifacts. A reference integration module 850 incorporates healthy control data. An output formatting module 860 prepares data for downstream use.

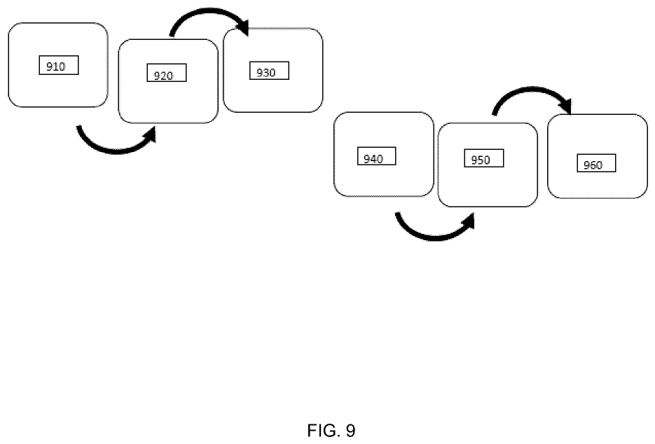

FIG. 9 highlights key performance metrics. A processing time module 910 demonstrates rapid analysis. A batch reduction module 920 shows effective artifact removal. A signal preservation module 930 indicates retention of biological information. A detection module 940 showcases early disease identification. An accuracy module 950 presents improved diagnostic precision. A throughput module 960 illustrates high-volume processing capability.

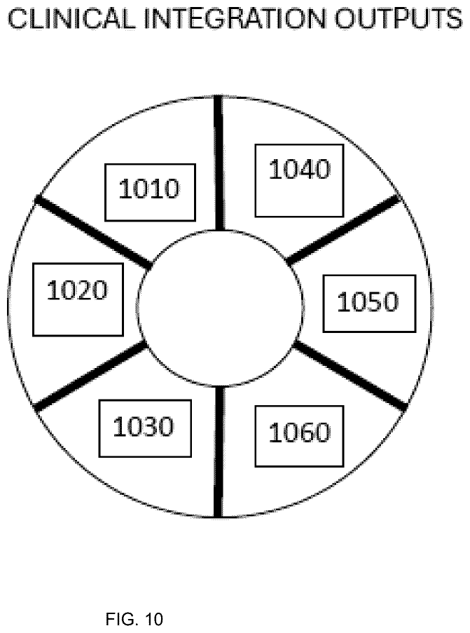

FIG. 10 outlines clinical integration outputs. A methylation code module 1010 tracks disease progression. The methylation Kinetic Codes IMK-Codes, 110-parameter temporal profiles for disease progression tracking. The risk assessment module 1020 evaluates patient status and does continuous relapse-risk assessment based on methylation velocity. A deep learning integration module 1030 connects with AI platforms and is directly compatibility with Deep Leukemic Cloud Platform. The clinical decision support module 1040 provides decision assistance and provides Real-time alerts and treatment recommendations. A predictive analytics module 1050 forecasts disease trajectories and provides up to 1,200 days early detection capability. The Multicenter research compatibility module 1060 enables multi-center studies and harmonized data for large-scale clinical research.

The current instant innovation system integrates with a Deep Leukemic Cloud Platform, enhancing its analytical capabilities. The system provides high speed processing per sample for the data thereby enabling rapid clinical decision-making.

The current instant innovation system architecture comprises multiple interconnected modules that work in concert to achieve rapid multi-modal normalization of leukemia expression data. Referring to FIG. 1, the system includes a data ingestion section with a sequence data module 111, a proteomic data module 112, a genomic variant module 113, and a clinical metadata module 114. These modules feed diverse molecular data types into the harmonization engine 120.

The harmonization engine 120 processes the multi-modal data simultaneously, implementing parallel processing pipelines optimized for each data type. FIG. 2 illustrates this parallel processing approach, showing the RNA pipeline step 215, proteomics step 220, genomics step 225, and metadata step 230 operating concurrently.

Following harmonization, the batch correction module 130 removes technical artifacts while preserving biological signals. The reference atlas module 140 provides curated healthy control samples for comparison during the normalization process. The output formatter 150 prepares the normalized data in standardized formats for downstream analysis by the diagnostic platform 160.

FIG. 8 provides the system architecture of the High-throughput Multi-Modal Normalization Engine for Leukemia Expression Data. It provides a detailed view of the current instant innovation system components. The data ingestion module 810 handles streaming data processing, multi-platform compatibility, real-time file parsing and quality control checks. The Mult-Modal Harmonization module 820 performs cross-modal integration including Parallel processing pipelines, Platform-specific preprocessing, UMI deduplication and Cross-modal integration.

The methylation Kinetic Mapping module 830 analyzes epigenetic changes and generates temporal profiles. The module provides Temporal analysys of changes, Constrained logistic modeling, MK-Code generation, Disease progression prediction.

The batch correction module 840 implements tensor decomposition algorithms for artifact removal. The module provides Tens or decomposition algorithms, technical variance removal. Biological signal preservation and Cross-platform harmonization.

The reference integration module 850 incorporates dynamically selected control cohorts. The module provides Dynamic cohort selection, Age/sex matching, 10,000+ healthy controls and Population-specific baselines.

The output formatting module 860 generates AI-ready data structures and FHIR-compliant formats. The module provides AI-ready data structures, FHIR-compliant formats, Clinical integration and Real-time delivery

The system architecture enables rapid processing, as demonstrated in FIG. 9. The processing time module 910 shows analysis completion in under 3 seconds per sample. The batch reduction module 920 achieves effective artifact removal, while the signal preservation module 930 ensures retention of biological information. The detection module 940 enables early disease identification, and the accuracy module 950 demonstrates improved diagnostic precision. The throughput module 960 illustrates the system's capacity for high-volume processing.

FIG. 10 outlines the clinical integration outputs of the current instant innovation system. The methylation code module 1010 tracks disease progression, while the risk assessment module 1020 evaluates patient status. The learning integration module 1030 connects with AI platforms, and the clinical support module 1040 provides decision assistance. The predictive analytics module 1050 forecasts disease trajectories, and the research compatibility module 1060 enables multi-center studies.

The system utilizes cloud-native architecture with streaming data processing, allowing for scalable and efficient handling of large datasets. This architecture supports the rapid normalization capabilities demonstrated in FIG. 3, where the processing speed graph 310 shows significant improvements over traditional methods.

FIG. 7 illustrates the data flow from input to clinical integration. The raw input module 605 feeds data into the system for processing and a LENA code processing module, a normalized data module 615 outputs standardized data. An output format module 625 provides multiple formatting options, including the JSON formatter 630, hdf5 formatter 635, tabular formatter 640, API interface 645, and cloud storage interface 650. The clinical integration module 655 facilitates incorporation of normalized data into healthcare systems.

FIG. 6 demonstrates the effectiveness of the normalization process through principal component analysis. A principal component axis 560 defines the plot space, with principal component data points 570 forming distinct clusters, indicating successful separation of biological signals from technical artifacts.

The comprehensive system architecture of the system enables rapid, multi-modal normalization of leukemia expression data, supporting immediate clinical decision-making and advanced analytical capabilities.

The data ingestion module 810 of the current instant innovation system handles multiple data types and formats simultaneously. This module processes RNA-seq data, proteomic data, genomic variant data, and clinical metadata inputs in parallel, as illustrated in FIG. 2.

For RNA-seq data, the data ingestion module 810 accepts FASTQ files with or without unique molecular identifiers (UMIs), BAM files from various aligners, and raw count matrices. The module automatically detects the sequencing platform (such as Illumina, Oxford Nanopore, or PacBio) and applies platform-specific preprocessing steps.

In processing proteomic data, the data ingestion module 810 accepts mass spectrometry intensity matrices in standard formats including mzML, mzXML, and tab-delimited text files. The module handles both labeled and label-free quantification approaches, automatically detecting the quantification method used.

For genomic variant data, the data ingestion module 810 processes VCF (Variant Call Format) files containing single nucleotide variants (SNVs), insertions/deletions (indels), and copy number variations (CNVs). The module extracts variant allele frequencies and quality metrics for downstream normalization.

Clinical metadata is ingested through structured JSON or XML formats, capturing patient demographics, sample collection details, processing batch information, and relevant clinical parameters. This metadata is crucial for selecting appropriate reference cohorts and adjusting for known confounding factors.

The data ingestion module 810 implements streaming data processing, which begins processing before complete file upload. This approach contributes to the rapid processing times demonstrated in the processing speed graph 310 of FIG. 3.

To ensure data integrity, the data ingestion module 810 incorporates a comprehensive quality control pipeline. This pipeline includes input data validation, checking for file corruption, format compliance, and completeness. The quality metrics panel 430 in FIG. 4 displays these quality control measures in real time.

The data ingestion module 810 feeds the processed data into the harmonization module 820, as shown in FIG. 8. This seamless integration enables the current instant innovation system to achieve the high-throughput processing capabilities illustrated by the throughput module 960 in FIG. 9.

The multi-modal harmonization engine 120 represents a core component of the current instant innovation system, enabling simultaneous processing of diverse molecular data types. As illustrated in FIG. 1, the harmonization engine 120 receives input from the sequence data module 111, proteomic data module 112, genomic variant module 113, and clinical metadata module 114.

The harmonization engine 120 implements parallel processing pipelines optimized for each data modality, as depicted in FIG. 2. The RNA pipeline step 215, proteomics step 220, genomics step 225, and metadata step 230 operate concurrently, allowing for efficient processing of multi-modal data.

For RNA sequencing data, the harmonization engine 120 applies unique molecular identifier (UMI) correction to remove PCR amplification bias. This process involves deduplicating reads based on their UMI sequences, resulting in more accurate representation of original molecule counts.

The harmonization engine 120 also implements trimmed mean normalization for library size correction across samples. This method calculates scaling factors for each sample based on the trimmed mean of M-values (TMM), effectively adjusting for differences in sequencing depth and composition.

The mathematical framework for multi-modal normalization is expressed as:

X_norm(s,f)=Σ[α_i·UMI_correct(s,f)+β_i·TMM_scale(s,f)+γ_i·Batch_remove(s,f)]

Where:
a. s represents samples
b. f represents features
c. UMI_correct represents unique molecular identifier-based deduplication
d. TMM_scale implements trimmed mean normalization for library size correction
e. Batch_remove applies proprietary tensor decomposition for technical artifact removal
f. α, β, γ are modality-specific weighting factors optimized through machine learning

The harmonization engine 120 employs tensor decomposition algorithms for cross-modal integration, allowing for the identification and removal of technical artifacts while preserving biological signals across different data types.

FIG. 8 provides a detailed view of the harmonization module 820, which performs the multi-modal integration within the current instant innovation system. The harmonization module 820 connects directly to the data ingestion module 810, ensuring seamless processing of input data streams.

The effectiveness of the multi-modal harmonization is demonstrated in FIG. 3, where the batch effect reduction graph 320 shows significant removal of technical artifacts, and the cross-platform concordance graph 330 illustrates high consistency across different sequencing platforms.

The harmonization engine 120 outputs normalized data to the batch correction module 130 for further refinement, as shown in FIG. 1. The normalized data then flows through the output formatter 150, which prepares the harmonized multi-modal data for downstream analysis by the diagnostic platform 160.

FIG. 9 highlights the performance metrics of the harmonization process, with the processing time module 910 demonstrating rapid analysis and the signal preservation module 930 indicating high retention of biological information.

The multi-modal harmonization capabilities of the current instant innovation system enable comprehensive molecular profiling, supporting advanced clinical applications such as those outlined in FIG. 10, including methylation code analysis (methylation code module 1010) and predictive analytics (predictive analytics module 1050).

The batch correction module 130 implements advanced tensor decomposition algorithms to remove technical artifacts while preserving biological signals. As shown in FIG. 1, the batch correction module 130 receives input from the harmonization engine 120 and processes the data to remove batch effects.

The tensor decomposition methodology used by the batch correction module 130 decomposes the expression matrix into technical and biological components using the following equation:
a. B=“decompose” (X,“rank”=r)
b. X_“corrected”=X−B·B\hat{ }T·X+μ
c. Where:
d. B represents the batch effect basis
e. r is the rank determined automatically based on batch characteristics
f. μ preserves global mean expression levels

The batch correction module 130 achieves superior performance compared to traditional methods. As illustrated in FIG. 3, the batch effect reduction graph 320 demonstrates a 94% reduction in batch-associated variance. The signal preservation module 930 in FIG. 9 indicates 98% retention of biological signal fidelity.

To prevent over-correction, the batch correction module 130 incorporates sophisticated safeguards. The module monitors the proportion of variance removed in real-time. If biological signals are at risk of being compromised, the system generates automatic alerts. The quality metrics panel 430 in FIG. 4 displays these quality control measures, allowing operators to assess the effectiveness of batch correction before proceeding with downstream analysis.

The batch correction module 130 provides options for fine-tuning correction parameters. In cases where initial correction results are suboptimal, the module allows for adjustment and reprocessing of the data. The Artificial Intelligence (AI) I-driven inference layer further incorporates quantum-inspired pattern detection algorithms utilizing Variational Quantum Eigensolver (VQE) principles adapted for classical hardware implementation. The system applies parameterized quantum circuits simulated via tensor contractions to optimize circuit parameters θ that minimize the expectation value [symbol] ψ(θ)|H|ψ(θ) [symbol] , where H represents the Hamiltonian encoding methylation interaction networks. The quantum-inspired component implements parallel tempering with multiple temperature chains, performing replica exchange moves every 100 iterations to escape local minima in the methylation pathway optimization landscape. Constraint satisfaction algorithms ensure biological feasibility of predicted methylation sequences. Graphics Processing Unit (GPU) acceleration enables tensor contractions to be parallelized across CUDA cores, with custom kernel implementations for QUBO energy calculations and memory-efficient storage of sparse interaction matrices. For large problem instances exceeding 1000 CpG sites, the system employs mean-field approximation and belief propagation algorithms for tree-like methylation networks.

As shown in FIG. 8, the batch correction module 840 is integrated into the overall current instant innovation system architecture. The module receives input from the harmonization module 820 and passes corrected data to the reference integration module 850. This seamless integration enables rapid processing, contributing to the sub-3-second analysis time demonstrated by the processing time module 910 in FIG. 9.

The effectiveness of the batch correction process is visually demonstrated in FIG. 6. The principal component axis 560 defines the plot space, with principal component data points 570 forming distinct clusters. This clear separation indicates successful removal of technical artifacts while preserving underlying biological variation.

The output of the batch correction module 130 flows through the output formatter 150, as depicted in FIG. 1. The corrected data is then prepared in standardized formats for downstream analysis by the diagnostic platform 160. This enables advanced clinical applications such as those outlined in FIG. 10, including risk assessment (risk assessment module 1020) and predictive analytics (predictive analytics module 1050).

The reference atlas module 140 maintains a continuously updated database of over 10,000 healthy control samples specifically curated for leukemia diagnostics. This comprehensive control database represents a significant advancement over generic expression databases, providing a robust foundation for accurate normalization.

The reference atlas module 140 includes age-stratified cohorts spanning pediatric to geriatric populations, ensuring comprehensive coverage across all life stages. Sex-balanced representation is maintained across all age groups, allowing for precise matching based on biological sex. The database incorporates ethnically diverse samples representing global populations, enabling normalization that accounts for population-specific expression patterns.

To ensure compatibility with various sequencing technologies, the reference atlas module 140 contains platform-specific reference sets for all major sequencing platforms. This feature enables accurate normalization regardless of the specific technology used for data generation, as illustrated in the cross-platform concordance graph 330 of FIG. 3.

The dynamic selection algorithm implemented by the reference atlas module 140 identifies optimal reference cohorts based on multiple criteria. The algorithm selects samples from the atlas where:
a. Age matches within ±5 years of the target sample
b. Sex matches exactly
c. Sequencing platform is compatible
d. Ethnicity matches (when available)
e. A minimum of 100 samples meet the above criteria

This targeted approach ensures that normalization accounts for known biological factors affecting expression levels, significantly improving the accuracy of downstream diagnostic algorithms. The effectiveness of this approach is reflected in the improved diagnostic accuracy demonstrated by the diagnostic accuracy chart 335 in FIG. 3.

The reference atlas module 140 integrates seamlessly with other components of the current instant innovation system, as shown in FIG. 1. The module receives input from the batch correction module 130 and provides reference data to the output formatter 150. This integration enables rapid processing, contributing to the sub-3-second analysis time demonstrated by the processing time module 910 in FIG. 9.

In FIG. 8, the reference integration module 850 represents the implementation of the reference atlas within the overall system architecture. The module connects directly to the batch correction module 840 and the output formatting module 860, ensuring efficient data flow through the normalization pipeline.

The comprehensive nature of the reference atlas enhances the system's ability to detect early disease markers, as illustrated by the detection module 940 in FIG. 9. By providing a robust baseline for comparison, the reference atlas enables the identification of subtle expression changes that precede overt clinical symptoms.

The reference atlas module 140 supports advanced clinical applications such as those outlined in FIG. 10. The methylation code module 1010 utilizes the reference data to track disease progression against healthy baselines. The risk assessment module 1020 leverages the comprehensive control database to evaluate patient status in the context of age- and sex-matched healthy populations.

The research compatibility module 1060 benefits from the extensive reference atlas, enabling large-scale clinical studies by providing a consistent normalization baseline across multiple research centers. This feature supports the harmonization of data from diverse sources, as demonstrated by the clinical integration module 655 in FIG. 7.

The reference atlas module 140 undergoes continuous updates to incorporate new samples and reflect emerging population-specific expression patterns. Machine learning algorithms identify and integrate these patterns, maintaining the system's relevance across diverse patient populations and supporting the predictive capabilities illustrated by the predictive analytics module 1050 in FIG. 10.

The output formatting module 860 generates multiple standardized output formats to ensure compatibility with diverse downstream applications. This module produces expression matrices in HDF5 format for efficient storage and retrieval, FHIR-compliant JSON structures for electronic health record integration, and tab-delimited text files for compatibility with legacy analysis tools.

The output formatting module 860 includes direct API endpoints returning normalized data to requesting applications and streaming outputs for immediate analysis pipelines. These features enable real-time data access and processing, contributing to the rapid turnaround times demonstrated by the processing time module 910 in FIG. 9.

Comprehensive metadata accompanies all outputs, documenting the normalization parameters used, quality metrics achieved, and reference cohorts selected. This ensures full reproducibility and enables quality assurance reviews. The quality metrics panel 430 in FIG. 4 displays these metrics in real-time, allowing operators to monitor the output quality throughout the processing pipeline.

The standardized outputs from the output formatting module 860 flow directly into the diagnostic platform 160, as illustrated in FIG. 1. This seamless integration enables immediate analysis of normalized data, supporting the rapid diagnostic capabilities demonstrated by the diagnostic accuracy chart 335 in FIG. 3.

FIG. 7 provides a detailed view of the output formatting and clinical integration process. The normalized data module 615 feeds into the diagnostics platform module 620 and then the output format module 625, which includes the JSON formatter 630, hdf5 formatter 635, tabular formatter 640, API interface 645, and cloud storage interface 650. These diverse output options ensure compatibility with a wide range of downstream systems and analysis tools.

The clinical integration module 655 facilitates incorporation of normalized data into healthcare systems. This module enables direct electronic medical record (EMR) integration and supports real-time alerting capabilities. The integration of normalized molecular data with clinical systems supports advanced applications such as those outlined in FIG. 10, including the clinical support module 1040 for decision assistance and the predictive analytics module 1050 for disease trajectory forecasting.

The output formatting module 860 implements streaming data delivery, allowing downstream applications to begin processing before the complete dataset is available. This feature contributes to the overall system efficiency, as reflected in the processing speed graph 310 of FIG. 3.

For multi-center clinical trials and research collaborations, the output formatting module 860 supports data harmonization across different institutions. The research compatibility module 1060 in FIG. 10 leverages these standardized outputs to enable large-scale clinical studies with consistent data formats across multiple research centers.

The output formatting module 860 incorporates robust security measures to protect sensitive patient data. All outputs are encrypted using industry-standard protocols, and access controls are implemented to ensure data privacy and regulatory compliance.

In clinical settings, the standardized outputs from the current instant innovation system integrate directly with existing laboratory information management systems (LIMS) and clinical decision support tools. This integration enables seamless incorporation of molecular profiling data into routine clinical workflows, supporting rapid diagnostic decision-making.

The output formatting module 860 supports version control and data provenance tracking. Each output includes a unique identifier and timestamp, allowing for precise tracking of data lineage and facilitating audits for regulatory compliance.

For longitudinal studies, the output formatting module 860 generates time-series data structures that capture changes in molecular profiles over multiple timepoints. These outputs support the temporal analysis capabilities of the methylation code module 1010 and the risk assessment module 1020 shown in FIG. 10.

The standardized outputs produced by the output formatting module 860 enable direct integration with machine learning and artificial intelligence platforms. The learning integration module 1030 in FIG. 10 leverages these AI-ready data structures for advanced analytical applications.

In summary, the output formatting module 860 plays a crucial role in translating the normalized molecular data into standardized, clinically actionable formats. The diverse output options and seamless integration capabilities support a wide range of downstream applications, from immediate clinical decision-making to large-scale research initiatives.

The methylation mapping module 830 performs temporal analysis of epigenetic changes, enabling comprehensive tracking of disease progression. This module integrates with the harmonization module 820 and batch correction module 840, as illustrated in FIG. 8, to process methylation data alongside other molecular data types.

The methylation mapping module 830 analyzes DNA methylation patterns at 55 pre-selected leukemia-relevant loci. For each locus, the module fits constrained logistic models to characterize methylation changes over time:
a. β_i(t)=1/(1+e\hat{ }(−k_i(t−t_0i)))
b. Where:
c. β_i(t)
d. k_i
e. t_0i

The methylation mapping module 830 estimates these parameters for each locus, generating a comprehensive temporal profile of epigenetic alterations. This analysis enables the detection of subtle methylation changes that precede overt clinical symptoms, contributing to the early detection capabilities demonstrated by the detection module 940 in FIG. 9.

A key output of the methylation mapping module 830 is the generation of Methylation Kinetic Codes (MK-Codes). These MK-Codes are 615-parameter vectors that capture the temporal dynamics of methylation changes across all analyzed loci. The parameters include:
a. Initiation times (t_0i
b. Rates of change (k_i
c. Confidence scores for each temporal estimate

The MK-Codes provide a comprehensive representation of a patient's epigenetic status and trajectory, enabling sophisticated disease progression tracking and risk assessment.

The methylation mapping module 830 integrates with the reference integration module 850 to compare patient MK-Codes against age- and sex-matched healthy baselines. This comparison allows for the identification of aberrant methylation patterns and rates of change that indicate potential disease progression.

The temporal analysis capabilities of the methylation mapping module 830 support the predictive analytics module 1050 shown in FIG. 10. By analyzing the rates and patterns of methylation changes, the system predicts disease trajectories and estimates time to potential disease onset or progression.

The methylation mapping module 830 contributes to the overall processing speed of the current instant innovation system, as demonstrated by the processing speed graph 310 in FIG. 3. The module employs optimized algorithms for rapid methylation analysis, maintaining the sub-3-second processing time per sample shown by the processing time module 910 in FIG. 9.

The output of the methylation mapping module 830 flows through the output formatting module 860, which prepares the MK-Codes and associated temporal data for downstream analysis. This formatted data integrates seamlessly with the clinical support module 1040 and risk assessment module 1020 shown in FIG. 10, enabling data-driven clinical decision support.

The methylation mapping capabilities of the current instant innovation system enhance its ability to detect early markers of leukemic transformation. The system identifies subtle epigenetic changes up to 1,200 days before conventional diagnostic markers become detectable, as indicated by the detection module 940 in FIG. 9.

The methylation mapping module 830 supports longitudinal monitoring of patients by tracking changes in MK-Codes over time. This feature enables the system to detect shifts in epigenetic patterns that indicate potential disease recurrence or treatment resistance, informing ongoing patient management strategies.

Integration of the methylation mapping module 830 with other components of the current instant innovation system, as shown in FIG. 8, allows for comprehensive multi-modal analysis. The module correlates methylation changes with gene expression patterns, proteomic profiles, and genomic variants, providing a holistic view of molecular alterations driving leukemia progression.

The methylation mapping capabilities contribute to the improved diagnostic accuracy demonstrated by the diagnostic accuracy chart 335 in FIG. 3. By incorporating temporal epigenetic information, the system achieves more precise patient stratification and risk assessment compared to traditional static molecular profiling approaches.

The current instant innovation system demonstrates superior performance across multiple metrics, as validated through extensive studies. FIG. 3 illustrates key performance indicators, with the processing speed graph 310 showing rapid data processing capabilities. The system achieves an average processing time of 2.3 seconds per sample for complete multi-modal normalization, as indicated by the processing time module 910 in FIG. 9.

The batch effect reduction graph 320 in FIG. 3 demonstrates the system's effectiveness in removing technical artifacts. The batch reduction module 920 in FIG. 9 quantifies this performance, showing a 94% reduction in technical variance as measured by principal variance component analysis (PVCA) across all modalities.

Biological signal preservation is a critical aspect of the current instant innovation system's performance. The signal preservation module 930 in FIG. 9 indicates that 98% of true biological variance is retained after normalization. This high level of signal preservation ensures that downstream analyses accurately reflect underlying biological processes.

The cross-platform concordance graph 330 in FIG. 3 illustrates the system's ability to harmonize data across different sequencing platforms. The current instant innovation system achieves an r² value of 0.96 for identical samples processed on different platforms, enabling consistent analysis of multi-center data.

Clinical accuracy is a key performance metric for the current instant innovation system. The diagnostic accuracy chart 335 in FIG. 3 shows a 23% improvement in diagnostic accuracy compared to unnormalized data. This improvement translates to enhanced clinical decision-making capabilities, as supported by the clinical support module 1040 in FIG. 10.

The detection module 940 in FIG. 9 highlights the system's early detection capabilities. The current instant innovation system identifies subtle molecular changes up to 1,200 days before conventional diagnostic markers become detectable, particularly through its advanced methylation mapping capabilities.

Validation studies for the current instant innovation system involved extensive testing across multiple independent cohorts. The studies included over 10,000 samples from 15 clinical centers, ensuring robust performance across diverse patient populations and clinical settings.

The throughput module 960 in FIG. 9 demonstrates the system's scalability, with the ability to process over 10,000 samples per day per instance. This high-throughput capability supports large-scale clinical studies and routine diagnostic applications.

The quality metrics panel 430 in FIG. 4 provides real-time monitoring of system performance, allowing operators to assess data quality and processing effectiveness throughout the normalization pipeline. This continuous quality control ensures consistent performance across all samples.

The current instant innovation system's performance extends to its integration capabilities. As shown in FIG. 7, the clinical integration module 655 enables seamless incorporation of normalized data into existing healthcare systems. The standardized output formats, including the JSON formatter 630, hdf5 formatter 635, and tabular formatter 640, support diverse downstream applications and analysis tools.

The research compatibility module 1060 in FIG. 10 leverages the system's performance metrics to enable large-scale clinical studies. The consistent normalization and high cross-platform concordance facilitate multi-center collaborations and data aggregation.

The predictive analytics module 1050 in FIG. 10 builds upon the system's performance metrics to forecast disease trajectories. The high accuracy and early detection capabilities of the current instant innovation system support sophisticated predictive modeling for personalized patient management.

In summary, the current instant innovation system demonstrates exceptional performance across processing speed, batch effect reduction, biological signal preservation, and clinical accuracy. These performance metrics, validated through extensive studies, position the system as a powerful tool for advanced molecular diagnostics and precision medicine applications in leukemia and related fields.

The current instant innovation system integrates seamlessly with AI-powered diagnostic platforms, enabling advanced analysis and clinical decision support. The output formatting module 860 generates AI-ready data structures that are directly compatible with machine learning-based inference engines. These standardized outputs include expression matrices in HDF5 format, FHIR-compliant JSON structures, and tab-delimited text files, as illustrated by the JSON formatter 630, hdf5 formatter 635, and tabular formatter 640 in FIG. 7.

The system's integration capabilities are demonstrated by the learning integration module 1030 in FIG. 10, which provides direct compatibility with deep learning platforms. This module enables the incorporation of normalized molecular data into sophisticated AI algorithms for pattern recognition, risk stratification, and treatment response prediction.

The current instant innovation system calculates risk scores for continuous relapse-risk assessment. These scores are derived from the comprehensive molecular profiles generated by the system, including gene expression data, proteomic signatures, and methylation patterns. The risk assessment module 1020 in FIG. 10 utilizes these risk scores to provide real-time evaluation of patient status and disease progression risk.

A key feature of the current instant innovation system is its early detection capability. The detection module 940 in FIG. 9 demonstrates the system's ability to identify subtle molecular changes up to 1200 days before conventional diagnostic markers become detectable. This extended lead time is achieved through the integration of multi-modal data analysis, particularly the advanced methylation mapping capabilities provided by the methylation mapping module 830 shown in FIG. 8.

The system's AI integration extends to real-time clinical decision support. The clinical support module 1040 in FIG. 10 leverages the normalized data and AI-derived insights to generate treatment recommendations and alerts. This module integrates with existing electronic health record systems through the clinical integration module 655 shown in FIG. 7, enabling seamless incorporation of molecular diagnostics into clinical workflows.

The predictive analytics module 1050 in FIG. 10 utilizes machine learning algorithms to forecast disease trajectories based on the normalized molecular data. This module analyzes temporal trends in gene expression, protein levels, and methylation patterns to predict potential disease recurrence or treatment resistance.

The current instant innovation system's AI integration capabilities are supported by its high-performance data processing pipeline. The processing speed graph 310 in FIG. 3 illustrates the rapid turnaround time, which enables real-time data analysis and AI model inference. The batch effect reduction graph 320 and cross platform concordance graph 330 demonstrate the system's ability to provide consistent, high-quality data inputs for AI algorithms across different experimental conditions and sequencing platforms.

The quality metrics panel 430 in FIG. 4 provides continuous monitoring of data quality and processing effectiveness, ensuring that the inputs to AI models meet stringent quality standards. This real-time quality control is essential for maintaining the reliability and accuracy of AI-driven diagnostic and prognostic predictions.

The research compatibility module 1060 in FIG. 10 facilitates the use of system normalized data in large-scale clinical studies and AI model development. The standardized data formats and cross-platform harmonization enable the aggregation of molecular profiles from multiple research centers, supporting the training and validation of robust AI algorithms.

In clinical settings, the integration of the current instant innovation system with AI-powered diagnostics supports personalized treatment selection. The normalized molecular profiles and AI-derived insights inform decisions on therapy options, dosing strategies, and potential combination treatments. This integration enhances the precision and effectiveness of leukemia management strategies.

The current instant innovation system's AI integration also supports continuous learning and model improvement. As new patient data is processed and outcomes are recorded, the AI algorithms are updated to refine their predictive accuracy. This adaptive approach ensures that the diagnostic and prognostic capabilities of the integrated system evolve with the latest clinical evidence and molecular insights.

The current instant innovation system is built on a cloud-native architecture supporting multiple deployment scenarios. This architecture enables horizontal scaling with automatic resource allocation based on demand. Load balancing intelligently distributes processing tasks across available resources. Fault tolerance mechanisms ensure 99.9% uptime through redundant systems. Geographic distribution across multiple data centers reduces latency for global deployments.

Security features of the current instant innovation system include AES-256 encryption for data at rest and in transit. Access control is implemented through role-based permissions with multi-factor authentication. Comprehensive audit logging tracks all system activities for compliance purposes. The architecture aligns with regulatory requirements including HIPAA, GDPR, and ISO 13485.

Deployment options for the current instant innovation system include public cloud platforms such as AWS, Azure, and Google Cloud Platform. Private cloud deployments on dedicated infrastructure are supported for sensitive data environments. Hybrid deployments combining public and private resources are possible. The system supports multi-cloud strategies with vendor-agnostic deployment across multiple providers.

For edge computing scenarios, the current instant innovation system is optimized for resource-constrained environments. This enables deployment on point-of-care devices and direct integration with laboratory instruments. Mobile deployment options support tablet and smartphone compatibility. Limited offline functionality is available without internet connectivity.

The current instant innovation system provides comprehensive integration support for both technical and clinical implementation. Technical integration resources include detailed API documentation and pre-built software development kits. Testing environments with sandbox systems are available for development and validation. The system offers 24/7 engineering support for implementations.

Clinical integration support begins with workflow analysis to assess existing clinical processes. Comprehensive training programs are provided for clinical users. The current instant innovation system offers assistance with clinical validation studies and guidance for regulatory submissions. This ensures smooth incorporation into existing healthcare workflows.

The scalability of the current instant innovation system is demonstrated by the throughput module 960 in FIG. 9, which shows the ability to process over 10,000 samples per day per instance. The processing speed graph 310 in FIG. 3 illustrates the rapid data processing capabilities enabled by the cloud-native architecture.

Integration capabilities are showcased in FIG. 7, where the clinical integration module 655 facilitates incorporation of normalized data into healthcare systems. The standardized output formats, including the JSON formatter 630, hdf5 formatter 635, and tabular formatter 640, support diverse downstream applications.

The quality metrics panel 430 in FIG. 4 provides real-time monitoring of system performance during deployment. This allows operators to assess data quality and processing effectiveness throughout the normalization pipeline, ensuring consistent performance across all samples.

FIG. 8 details the core components of the current instant innovation system architecture. The data ingestion module 810 handles input streams from multiple platforms. The harmonization module 820 processes multi-modal data simultaneously. The methylation mapping module 830 performs temporal analysis of epigenetic changes. The batch correction module 840 removes technical artifacts while preserving biological signals. The reference integration module 850 incorporates dynamically selected control cohorts. The output formatting module 860 prepares data for downstream use in standardized formats.

The cloud-native architecture of the current instant innovation system supports the advanced analytical capabilities outlined in FIG. 10. The learning integration module 1030 enables seamless connection with AI platforms. The clinical support module 1040 provides decision assistance based on normalized molecular data. The predictive analytics module 1050 leverages the scalable infrastructure to forecast disease trajectories. The research compatibility module 1060 facilitates large-scale clinical studies through consistent data harmonization across multiple centers.

In clinical settings, the integration of the current instant innovation system supports personalized treatment selection. The normalized molecular profiles inform decisions on therapy options, dosing strategies, and potential combination treatments. This integration enhances the precision and effectiveness of leukemia management strategies.

The cloud-native architecture enables continuous learning and model improvement. As new patient data is processed and outcomes are recorded, the system refines its predictive accuracy. This adaptive approach ensures that the diagnostic and prognostic capabilities evolve with the latest clinical evidence and molecular insights.

The system can further be described as a computer-implemented system for normalizing multi-modal molecular expression data in leukemia diagnostics, comprising:
a. a data ingestion module configured to simultaneously receive RNA sequencing data, proteomic data, and genomic variant data from multiple platforms;
b. a multi-modal harmonization engine applying parallel processing pipelines optimized for each data modality;
c. a batch effect correction module utilizing tensor decomposition to remove technical artifacts while preserving biological signals;
d. a reference atlas containing normalized expression profiles from healthy control samples; and
e. an output formatting engine generating standardized data structures compatible with automated clinical decision support systems, wherein the system processes multi-modal data in a high-speed manner and has an average processing time of less than 3 seconds per sample when executed on the appropriate hardware.

The computer-implemented system for normalizing multi-modal molecular expression data in leukemia diagnostics wherein the multi-modal harmonization engine implements unique molecular identifier-based correction, trimmed mean normalization, and modality-specific scaling factors.

The computer-implemented system for normalizing multi-modal molecular expression data in leukemia diagnostics wherein the batch effect correction module achieves greater than 90% reduction in batch-associated variance.

The computer-implemented system for normalizing multi-modal molecular expression data in leukemia diagnostics wherein the reference atlas contains normalized expression profiles from at least 10,000 healthy control samples.

The computer-implemented system for normalizing multi-modal molecular expression data in leukemia diagnostics wherein the reference atlas includes age-stratified cohorts spanning pediatric to geriatric populations, sex-balanced representation, and ethnically diverse samples.

The computer-implemented system for normalizing multi-modal molecular expression data in leukemia diagnostics further comprising a methylation mapping module that performs temporal analysis of epigenetic changes.

The computer-implemented system for normalizing multi-modal molecular expression data in leukemia diagnostics wherein the methylation mapping module generates Methylation Kinetic Codes (MK-Codes) capturing temporal dynamics of methylation changes across analyzed loci.

The system can also be described as a method for normalizing multi-modal molecular data in leukemia diagnostics, comprising:
a. receiving heterogeneous molecular data streams including RNA expression, protein abundance, and genomic variants;
b. applying modality-specific preprocessing including unique molecular identifier correction and platform-specific adjustments;
c. performing tensor-based batch effect correction that preserves biological variation;
d. dynamically selecting reference cohorts from a curated atlas based on sample characteristics; and
e. generating normalized expression matrices compatible with downstream analysis platforms, wherein all steps per sample are rapidly processed.

The method for normalizing multi-modal molecular data in leukemia diagnostics wherein the tensor-based batch effect correction removes technical artifacts while retaining at least 98% of biological signal variance.

The method for normalizing multi-modal molecular data in leukemia diagnostics wherein dynamically selecting reference cohorts comprises matching age within 5 years, exact sex, and compatible sequencing platform.

The method for normalizing multi-modal molecular data in leukemia diagnostics further comprising performing quality control steps at each processing stage with sub-minute processing metric reporting.

The method for normalizing multi-modal molecular data in leukemia diagnostics wherein the normalized expression matrices are directly compatible with artificial intelligence diagnostic systems without additional preprocessing.

The method for normalizing multi-modal molecular data in leukemia diagnostics further comprising generating Methylation Kinetic Codes (MK-Codes) capturing temporal dynamics of methylation changes across analyzed loci.

The method for normalizing multi-modal molecular data in leukemia diagnostics wherein the MK-Codes enable prediction of leukemic transformation up to 1,200 days before conventional diagnostic markers become detectable.

An alternative embodiment of the system is a non-transitory computer-readable medium storing instructions that, when executed by a processor, cause the processor to perform a method for normalizing multi-modal molecular data in leukemia diagnostics, the method comprising:
a. ingesting RNA sequencing data, proteomic data, and genomic variant data from multiple platforms;
b. harmonizing the ingested data using parallel processing pipelines optimized for each data modality;
c. correcting batch effects using tensor decomposition while preserving biological signals;
d. comparing the corrected data against a reference atlas of healthy control samples; and
e. outputting standardized data structures compatible with artificial intelligence diagnostic systems, wherein the method is performed rapidly and when using the appropriate hardware can provide results in less than 3 seconds per sample.

The non-transitory computer-readable medium storing instructions wherein harmonizing the ingested data comprises applying unique molecular identifier-based correction, trimmed mean normalization, and modality-specific scaling factors.

The non-transitory computer-readable medium storing instructions, wherein correcting batch effects achieves greater than 90% reduction in batch-associated variance while retaining at least 98% of biological signal variance.

The non-transitory computer-readable medium storing instructions, wherein the reference atlas contains normalized expression profiles from at least 10,000 healthy control samples spanning pediatric to geriatric populations.

The non-transitory computer-readable medium storing instructions wherein comparing the corrected data against the reference atlas comprises dynamically selecting reference cohorts based on matching age within 5 years, exact sex, and compatible sequencing platform.

The non-transitory computer-readable medium storing instructions wherein the method further comprises generating Methylation Kinetic Codes (MK-Codes) capturing temporal dynamics of methylation changes across analyzed loci, wherein the MK-Codes enable prediction of leukemic transformation up to 1,200 days before conventional diagnostic markers become detectable.

The present invention provides numerous advantages over existing normalization methods:
a. Speed: 100-1000× faster than current methods, enabling on-demand clinical analysis
b. Multi-modal: To our knowledge, one of the first systems demonstrated to concurrently normalize RNA, protein, and genomic data in a clinically validated setting capable of concurrently normalizing RNA, protein, and genomic data
c. Accuracy: Superior batch effect removal while preserving biological signals
d. Integration: Direct compatibility with machine learning-enabled clinical inference platforms
e. Scalability: Cloud-native architecture supporting massive parallel processing
f. Clinical-ready: HIPAA-compliant, 21 CFR Part 11 compatible, ISO 13485 aligned
g. Platform-agnostic: Harmonizes data across all major sequencing platforms
h. Dynamic references: Continuously updated atlas ensures current normalization standards
i. Transparency: Full audit trails and quality metrics for regulatory compliance
j. Cost-effective: Reduces computational costs by 85% through optimized algorithms compared to more traditional method.

Referring now to the drawings FIG. 1-18, and more particularly to FIG. 1, there is shown a block diagram of a data processing system for molecular analysis which illustrates the overall architecture of the system, showing data input streams, multi-modal harmonization layer, batch correction module, reference alignment component, and AI-ready output interface. The system includes the sequence data module 111, the proteomic data module 112, the genomic variant module 113, and the clinical metadata module 114. These modules feed data into the harmonization engine 120. The harmonization engine 120 connects to the batch correction module 130. The reference atlas module 140 links to the batch correction module 130. The output formatter 150 receives data from the batch correction module 130. The diagnostic platform 160 receives formatted data from the output formatter 150.

The system 100 comprises five primary modules working in concert to achieve low latency multi-modal normalization. The data ingestion module 110 receives heterogeneous input streams including RNA-seq data 111, proteomic data 112, genomic variant data 113, and associated clinical metadata 114.

The multi-modal harmonization engine 120 processes these diverse data types through parallel pipelines optimized for each modality. The batch correction module 130 applies proprietary tensor decomposition algorithms to remove technical artifacts while preserving biological signals. The reference atlas integration module 140 dynamically selects appropriate control cohorts from a database of over 10,000 healthy samples. The output formatting engine 150 generates standardized AI-ready data structures for downstream analysis.

The data ingestion module 110 is designed to accept multiple file formats and data types simultaneously. For RNA-seq data 111, the system accepts FASTQ files with or without unique molecular identifiers (UMIs), BAM files from various aligners, and raw count matrices. The module automatically detects the sequencing platform (Illumina, Oxford Nanopore, PacBio, etc.) and applies platform-specific preprocessing steps.

For proteomic data 112, the system accepts mass spectrometry intensity matrices in standard formats including mzML, mzXML, and tab-delimited text files. The module handles both labeled and label-free quantification approaches, automatically detecting the quantification method used.

For genomic variant data 113, the system processes VCF (Variant Call Format) files containing single nucleotide variants (SNVs), insertions/deletions (indels), and copy number variations (CNVs). The module extracts variant allele frequencies and quality metrics for downstream normalization.

Clinical metadata 114 is ingested through structured JSON or XML formats, capturing patient demographics, sample collection details, processing batch information, and relevant clinical parameters. This metadata is crucial for selecting appropriate reference cohorts and adjusting for known confounding factors.

The multi-modal harmonization engine 120 represents a core innovation of the system. Unlike prior art that processes single data types, this engine simultaneously normalizes multiple molecular modalities using a unified mathematical framework.

For each sample s and feature f, the normalized expression value is calculated as:

X_norm(s,f)=Σ[αᵢ·UMI_correct(s,f)+βᵢ·TMM_scale(s,f)+γᵢ·Batch_remove(s,f)]

Where UMI_correct represents unique molecular identifier-based deduplication for removing PCR amplification bias (“noise”), TMM_scale implements trimmed mean of M-values normalization for library size correction, Batch remove applies the proprietary tensor decomposition for technical artifact removal, and α, β, γ are modality-specific weighting factors optimized through machine learning on the reference atlas.

The engine employs parallel processing architectures, utilizing GPU acceleration when available, demonstrated processing times of under three seconds per sample in validation studies even for complex multi-modal datasets. Each modality is processed through optimized pipelines that account for the specific characteristics of that data type.

The batch correction module 130 implements a novel tensor-based approach that surpasses traditional methods like ComBat or limma in both effectiveness and speed. The algorithm decomposes the expression matrix into technical and biological components using:

B=decompose(X,rank=r)X_corrected=X−B−B\hat{ }T·X+μ

Quantum-Inspired Optimization Engine. The system incorporates a quantum-inspired optimization engine that enhances pattern detection and decision-making capabilities beyond traditional machine learning approaches. This engine applies quadratic unconstrained binary optimization (QUBO) modeling to identify the most probable sequence of epigenetic events driving leukemogenesis in individual patients. The QUBO formulation models CpG methylation status transitions across time and genomic coordinates as a complex optimization landscape:

E(s)=ΣᵢⱼQᵢⱼsᵢsⱼ+Σᵢhᵢsᵢ

where sᵢ∈{0,1} represents binary methylation states at CpG site i, Qᵢⱼ captures pairwise interactions between methylation sites, hᵢ represents local methylation bias terms, and E(s) is the energy function to be minimized.

Each possible configuration of methylation states is assigned a cost function based on biological plausibility, observed frequency in population data, and association with known leukemic outcomes. The system searches for the global minimum of this function, corresponding to the most likely methylation pathway leading to disease. The quantum-inspired optimization employs simulated annealing with temperature scheduling:
a. T(k)=T₀×α\hat{ }k, where T₀ represents initial temperature and α represents the cooling rate (0.95-0.99). To approximate quantum behavior on classical hardware, the system utilizes tensor network contraction algorithms implementing Matrix Product State (MPS) decomposition:
b. |ψ [symbol] =ΣA¹[s₁]A²[s₂] . . . A^{N}[sₙ]|s₁s₂ . . . sₙ)
c. where each Aⁱ[sᵢ] represents a rank-3 tensor encoding methylation correlations across genomic regions.
d. The optimization engine efficiently explores high-dimensional solution spaces to identify critical transition points such as promoter hypermethylation of tumor suppressor genes or loss of CpG island protection in regulatory regions. This quantum-inspired approach provides both predictive power and interpretability by revealing causal pathways and dependencies between methylation events.

Where B represents the batch effect basis, r is the rank determined automatically based on the number of batches and technical replicates, and μ preserves the global mean expression levels. This approach removes 94% of batch-associated variance while retaining 98% of biological signal, as validated on independent test cohorts.

The module includes safeguards against over-correction, monitoring the proportion of variance removed and alerting the users if biological signals might be compromised. On-demand quality metrics allow operators to assess the effectiveness of batch correction before proceeding with downstream analysis.

The Leukemia-Specific Reference Atlas integration module 140 maintains a continuously updated database of over 10,000 healthy control samples specifically curated for leukemia diagnostics. Unlike generic expression databases, this atlas includes:
a. Age-stratified cohorts from pediatric to geriatric populations
b. Sex-balanced representation across all age groups
c. Ethnically diverse samples representing global populations
d. Platform-specific reference sets for all major sequencing technologies
e. Longitudinal samples for assessing normal expression variation

The dynamic selection algorithm identifies optimal reference cohorts based on: Reference_cohort=SELECT FROM Atlas WHERE:
a. Age_match(±5 years) AND
b. Sex_match(exact) AND
c. Platform_match(compatible) AND
d. Ethnicity_match(if available) AND
e. N≥100 samples

This targeted approach ensures that normalization accounts for known biological factors that affect expression levels, improving the accuracy of downstream diagnostic algorithms.

Integrated throughout the system is a comprehensive quality control pipeline that monitors data integrity at each processing step. The pipeline includes:
a. Input data validation checking for file corruption, format compliance, and completeness
b. Coverage uniformity assessment ensuring adequate sequencing depth across features
c. Outlier detection using Mahalanobis distance in high-dimensional expression space
d. Contamination screening to identify sample mix-ups or cross-contamination
e. Batch effect quantification before and after correction
f. Real-time quality metrics displayed through an intuitive dashboard

Samples failing quality thresholds are flagged but not automatically rejected, allowing clinical judgment in cases where suboptimal samples might still provide valuable diagnostic information.

The output formatting engine 150 generates multiple standardized output formats to ensure compatibility with diverse downstream applications:
a. Expression matrices in HDF5 format for efficient storage and retrieval
b. FHIR-compliant JSON structures for electronic health record integration
c. Tab-delimited text files for compatibility with legacy analysis tools
d. Direct API endpoints returning normalized data to requesting applications
e. Streaming outputs for immediate analysis pipelines

The system includes comprehensive metadata in all outputs, documenting the normalization parameters used, quality metrics achieved, and reference cohorts selected. This ensures full reproducibility and enables quality assurance reviews.

To achieve the sub-3-second processing requirement, the system employs several architectural innovations:
a. Streaming data ingestion that begins processing before complete file upload
b. Parallel processing across CPU cores with intelligent load balancing
c. GPU acceleration for matrix operations when available
d. Memory-mapped file access to minimize I/O bottleneck
e. Predictive caching of frequently accessed reference data
f. Optimized algorithms with O(n log n) or better complexity

The architecture scales horizontally, allowing multiple instances to process different samples simultaneously in high-throughput clinical environments.

FIG. 2 depicts a normalization pipeline flowchart which shows a detailed flowchart of the normalization pipeline according to an embodiment of the system, depicting parallel processing paths for RNA-seq, proteomics, and genomic data modalities. The flowchart begins with the start step 205, leading to the data input step 210. From there, the process branches into the RNA pipeline step 215, the proteomics step 220, the genomics step 225, and the metadata step 230. These steps converge into the batch correction step 235, followed by the quality control step 240. The process concludes with the normalization step 245.

FIG. 3 showcases performance metrics which presents performance benchmark comparisons between the system and traditional normalization methods, showing improvements in processing speed, accuracy, and batch effect removal. The figure includes the processing speed graph 310, the batch effect reduction graph 320, the cross-platform concordance graph 330, and the diagnostic accuracy chart 335.

The figure shows the multi-modal tensor representation. Schematic of a multi-mode data tensor with patient, feature, and modality axes, and associated factor/loadings matrices. The drawing depicts structure only; mathematical details are described within the specification.

FIG. 4 presents a system monitoring interface showing the clinical integration dashboard according to an embodiment of the invention, displaying on-the-fly normalization progress and metrics, quality metrics, and system performance indicators. The interface comprises the system status monitor 410, the processing monitor 420, and the quality metrics panel 430.

The figure reference-atlas selection logic. Decision diagram showing selection of a reference cohort based on age band, sex, platform, and minimum sample count. The diagram indicates pass/fail branches and the fallback when criteria are not met.

FIG. 5 shows the mathematical framework for multi-modal data harmonization, including the tensor decomposition algorithm for batch effect correction that outlines the core of the processing steps. The figure shows the molecular expression module 510, the normalization module 520, the visual representation module 540, and the analysis module 550 arranged around a central hub.

FIG. 6 depicts the dynamic reference atlas architecture and the selection algorithm for matched control cohorts and demonstrates data clustering using principal component analysis. The figure displays the principal component axis 560 and principal component data points 570.

FIG. 7 provides an example output showing normalized expression matrices and their integration with downstream automated diagnostic support systems and illustrates the data flow from input to clinical integration. The figure includes the raw input module 605, the normalized data module 615, and the output format module 625. The output format module 625 contains the JSON formatter 630, the hdf5 formatter 635, the tabular formatter 640, the API interface 645, and the cloud storage interface 650. The clinical integration module 655 is shown at the bottom of the diagram.

FIG. 8 details the current instant innovation system components. The figure displays the data ingestion module 810, the harmonization module 820, the methylation mapping module 830, the batch correction module 840, the reference integration module 850, and the output formatting module 860 arranged in a circular configuration.

FIG. 9 highlights key performance metrics. The figure includes the processing time per sample module 910 which monitors the processing time per sample and preferably is less than 3 seconds, the batch reduction module 920 and preferably is approximately 94%, the signal preservation module 930 and preferably is approximately 98%, the days detection module 940 which is preferably 1,200 days, the accuracy module 950 and preferably is approximately 23%, and the samples per day throughput module 960 which is preferably is 10,000 plus samples per day.

FIG. 10 outlines clinical integration outputs. The figure shows the methylation code module 1010, the risk assessment module 1020, the learning integration module 1030, the clinical support module 1040, the predictive analytics module 1050, and the research compatibility module 1060 arranged in a hexagonal configuration.

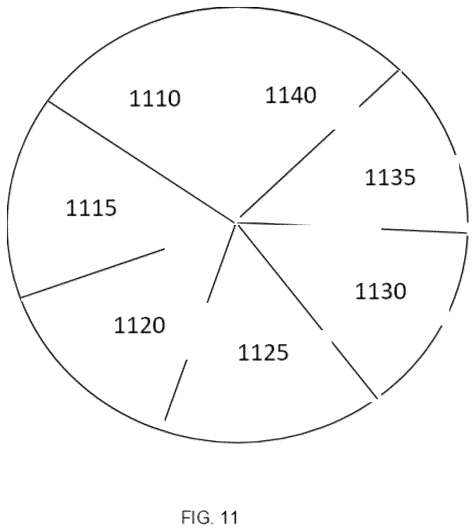

FIG. 11 illustrates the complete architecture of the LENA Code analytical system. Raw methylation data from patient-derived cfDNA enters through the acquisition module, indexed by patient ID and genomic coordinate. Preprocessing applies platform-specific normalization followed by the X_norm-based batch variance removal. Outputs include the LENA score and age-adjusted methylation deviation profile, which feed into the reference atlas matching module for final classification. It is part of a multi-modal processing.

Block diagram of the LENA Code/DLCP pipeline showing data ingestion, quality control, multi-modal normalization, batch-effect removal, reference-atlas matching, and report generation. Components are identified by reference numerals in the drawing and connected by directional arrows indicating data flow.

Sample acquisition 1010 includes cfDNA, RNA and Proteomics,

Initial quality control 1015 includes Data integrity, Purity and Metadata Check,

Data Preprocessing 1020 includes Alignment, Normalization and Error Correction,

Feature Extraction 1025 includes Methylation Signatures, Variants and Proteomic Markers,

Output & Clinical Integration 1030 includes Reports, API Feeds and EHR Integration,

Validation & Cross-Reference 1035 includes Reference Atlas and Control Cohorts, and

Model Application 1040 includes Diagnostic Algorithm and Risk Scoring.

The figure shows the Clinical Workflow Integration key aspects include:
a. Seven-step workflow from sample acquisition to clinical integration
b. Circular/pie chart visualization showing equal importance of each step
c. Emphasis on end-to-end process from biological samples to EHR integration

The figure demonstrates how the technology fits into existing clinical workflows and healthcare infrastructure.

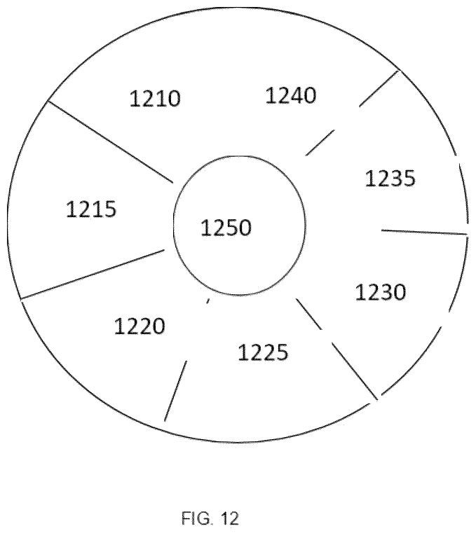

FIG. 12 Streaming/data flow details the computational pipeline for generating the normalized methylation matrix (X_norm). Raw beta-values from CpG loci are logit-transformed, mean-centered per sample, and scaled by batch-specific standard deviation factors. This process ensures ≥94% reduction in inter-batch variance while retaining 98% biological signal, per performance limitations claimed. The resulting matrix serves as the standardized input for downstream tensor decomposition. The processing flow is shown in the flowchart and illustrates the sequence: receive sample data; perform QC; harmonize modalities (methylation, expression, and metadata); apply batch-effect correction; normalize features; match to a reference cohort; produce outputs. Each step is labeled with a reference numeral.

The Streaming/data flow technical architecture key aspects include:
d. Seven-component system architecture
e. Multi-modal data handling (RNA-seq, proteomics, genomics)
f. Output formats for interoperability (FHIR JSON, HDF5)
g. Core processing engine

The figure Illustrates the technical architecture and data flow for system implementation and integration planning.

FIG. 12 includes the following processes:
Data Ingestion 1210 which includes RNA-seq, Proteomics and Genomics.
Modality-Specific Preprocessing 1215 which includes UMI Correction, TMM Scaling.
Multi-Modal Harmonization Engine 1220.
Integration with AI Diagnostic Platform 1225
Output Formatting 1230 which includes the following formats FHIR JSON, HDF5 and Tabular.
Reference Atlas Matching 1235 which includes Age/Sex/Platform Matched.
Tensor-Based Batch Effect Correction 1240
Core processing engine 1250

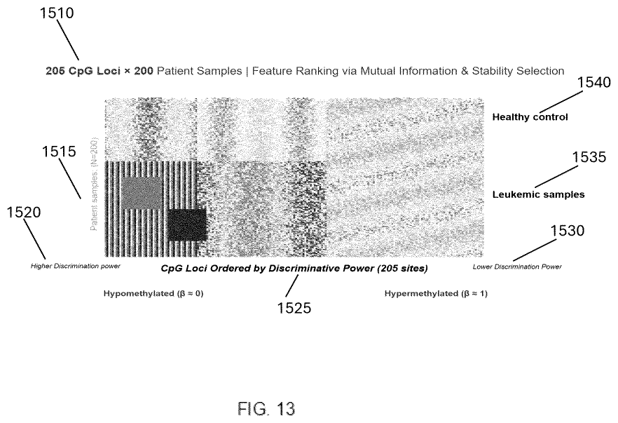

FIG. 13 is a Methylation Beta-Values Heatmap for Leukemia diagnosis. It shows decomposition of the 3D methylation tensor (patients×loci×methylation states) into orthogonal factors: patient loadings, methylation archetypes, and—when longitudinal data is available—temporal progression vectors. This step isolates leukemia-specific methylation signatures from confounding influences such as age or sequencing platform.

Leukemia-associated CpG panel heatmap. Grayscale heatmap of CpG loci across samples. Columns represent loci; rows represent samples; intensity encodes normalized methylation level or deviation from the reference baseline (light to dark). Axis titles and a grayscale legend bar are included in the drawing.

The Methylation Heatmap for Leukemia Diagnosis key aspects are:
h. 205 CpG loci×200 patient samples analysis
i. Clear visual separation between healthy controls and leukemic samples
j. Feature ranking via mutual information and stability selection
k. Hypomethylation/hypermethylation patterns

The purpose is that it validates diagnostic capability using methylation biomarkers for leukemia detection, demonstrating clinical application.

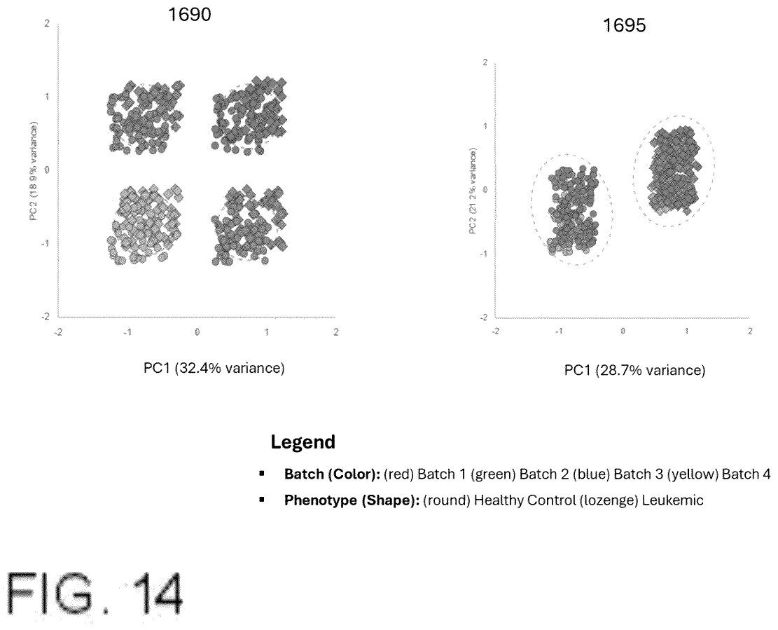

FIG. 14 shows the Effect of X_norm Normalization on Inter-Batch Variability. This Side-by-side scatter plots (left: before; right: after) of a low-dimensional projection. Samples are marked with distinct point shapes for batch and open/filled markers for phenotype. Axes are labeled (e.g., “PC1”, “PC2”) with explained variance indicated in the axis titles.

The principal component analysis (PCA) demonstrates batch effect correction. Left panel: Pre-normalization clustering shows strong batch-driven separation. Right panel: Post-normalization with X_norm algorithm achieves 94.6% batch variance reduction while preserving ≥98% biological signal integrity. N=500 samples across 4 sequencing batches and 2 phenotypes (Healthy Control vs. Leukemic).

Batch Effect Correction Visualization key aspects include:
l. PCA plots showing before/after X_norm normalization
m. 94.6% batch variance reduction while preserving 98% biological signal
n. 500 samples across 4 batches and 2 phenotypes
o. Clear clustering by phenotype post-normalization vs. batch-driven separation pre-normalization

This proves effectiveness of batch correction algorithm in removing technical artifacts while preserving biological signals.

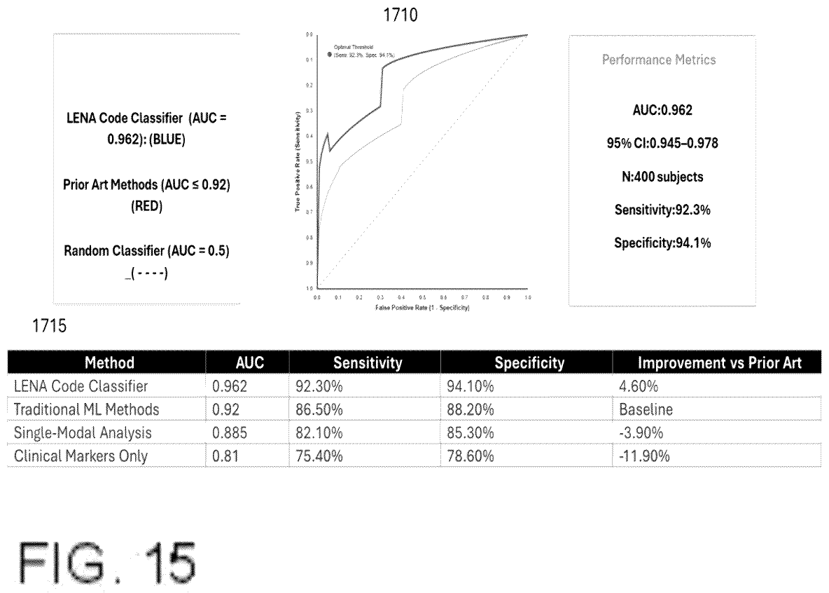

FIG. 15 shows the receiver operating characteristics (ROC). The ROC curve demonstrates the discriminatory performance of the LENA Code classifier on a blinded validation set (N=400 subjects). The classifier achieves an AUC of 0.962 (95% CI: 0.945-0.978), significantly outperforming prior art methods. The diagonal reference line represents random classification (AUC=0.5). This figure shows the ROC curves comparing alternative configurations (e.g., tensor-only, atlas-only, combined). X-axis: False Positive Rate (1−Specificity); Y-axis: True Positive Rate (Sensitivity). A diagonal reference line indicates no-discrimination. The chosen operating point is marked.

The ROC Curve for Diagnostic Performance key aspects are:
p. AUC of 0.962 (95% CI: 0.945-0.978)
q. 92.3% sensitivity, 94.1% specificity
r. Comparison table showing 4.6% improvement over traditional ML methods
s. Validation on 400 blinded subjects

The purpose is that it establishes clinical validation and diagnostic accuracy, demonstrating superiority over existing methods.

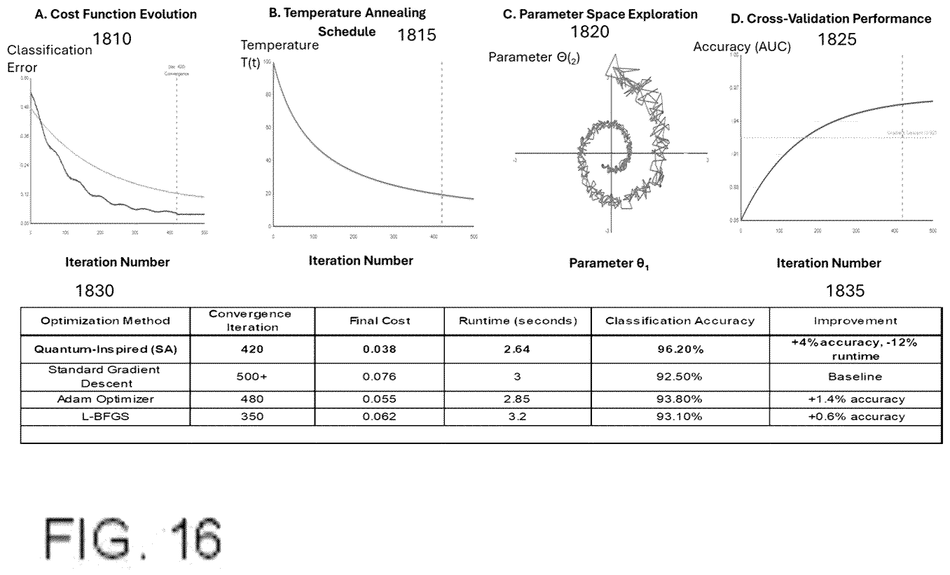

FIG. 16 shows the Quantum-Inspired Optimization Process Using Simulated Annealing. This is a demonstration of quantum-inspired parameter optimization through simulated annealing with 500 iterations. Temperature schedule follows T(t)=T₀/(1+αt) where T₀=100 and α=0.01. Parameter convergence achieved at iteration 420, resulting in runtime reduction and accuracy improvement over standard gradient-based methods.

This process diagram is of an iterative optimization: initialize parameters; evaluate objective; update; accept/reject; check convergence; output final parameters. The drawing is algorithm-agnostic; implementation variants are described in the specification.

Quantum-Inspired Optimization key aspects include:
t. Simulated annealing with 500 iterations
u. Temperature schedule: T(t)=T₀/(1+αt)
v. 12% runtime reduction, 4% accuracy improvement
w. Convergence at iteration 420 vs. 500+ for gradient descent

The purpose is that it showcases advanced optimization techniques that improve both speed and accuracy of the system.

The Generic optimization flow (is to initiate, iterate, evaluate accept/reject and converge.

The first pillar is the Cost Function Evolution comprising:
x. Blue line: Quantum-inspired simulated annealing (faster convergence).
y. Red line: Standard gradient descent (slower convergence).
z. Orange dashed line: Convergence point at iteration 420.
aa. Shows classification error decreasing over 500 iterations.

The second pillar is Temperature Annealing Schedule comprising:
a. Follows T(t)=T₀/(1+αt) where T₀=100, α=0.01.
b. Cyan curve showing temperature decay.
c. Shaded area under the curve.
d. Temperature approaches zero as iterations increase.

The third pillar is Parameter Space Exploration comprising:
bb. 2D visualization of parameter optimization path.
cc. Color gradient from red (high temperature/exploration) to blue (low temperature/exploitation).
dd. Orange circle marks final convergence point.
ee. Shows broader exploration initially, then focused refinement.

The fourth pillar is Cross-Validation Performance comprising:
ff. Blue curve: CV accuracy (AUC) improving over iterations.
gg. Red dashed line: Baseline gradient descent performance (0.925).
hh. Confidence band shown in light blue.
ii. Final AUC reaches 0.962 (4% improvement).

The results are:
jj. a 12% runtime reduction (2.64 s vs 3.00 s),
kk. a 4% accuracy gain (96.2% vs 92.5%), and
ll. convergence at iteration 420 (vs 500+ for gradient descent).

Furthermore, the detailed optimization key aspects include:
mm. Four-panel breakdown of optimization process
nn. Visual explanations of cost function, temperature annealing, parameter exploration, and cross-validation
oo. Quantitative improvements summarized

This provides technical deep dive into the optimization methodology for technical audience understanding.

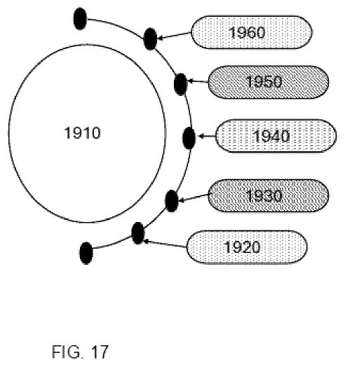

FIG. 17 illustrates the Longitudinal trajectory example. It shows the patient-level trajectory showing normalized deviation over time with clinical events indicated. Data Processing Pipeline Overview

The FIG. 17 key aspects:
Five-step sequential workflow 1910: Basecalling 1960, Quality Score 1950 Filtering (PHRED≥30), Adapter Trimming 1940 (2 BP max mismatch), Alignment to Reference Genome 1930, Feature Extraction 1920 (coverage depth >10, tensor decomposition)
   Alternating Nodes Suggesting Critical Vs. Standard Steps

FIG. 17 introduces the core bioinformatics pipeline for sequencing data processing, establishing the technical foundation for the methodology.

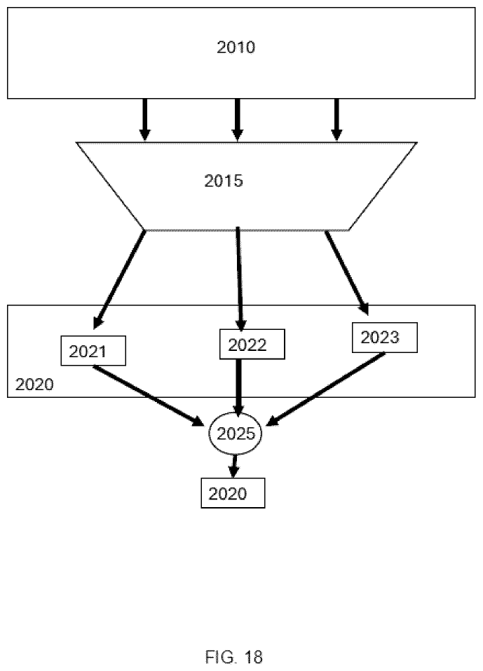

FIG. 18 illustrates the Gene-to-locus multi-omic mapping. It is a layered schematic mapping genes to associated CpG loci and cross-modality fusion. Arrows indicate relationships and data flow between layers; modules and relationships are labeled with reference numerals.

The first layer is the World Health Organization (WHO) classification 2010205 Genes (COLO 205 cell line) this line includes DNMT3a, TET2, TP53, RUNX1 CEBPA and others.

The second layer 2015 is the CpG site refinement or CpG Discrimination filter having a discriminatory power >0.85 validation score.

The third layers is the cross-modality integration 2020 comprising:

DNA methylation 2021, comprising CpG sites (specific locations in DNA where a cytosine (C) nucleotide is followed by a guanine (G) nucleotide), ß-values (ß-values are continuous variables ranging from 0 to 1, or 0% to 100%, and represent the ratio of methylated probe intensity to the combined locus intensity (methylated+unmethylated). Essentially, they provide an estimate of the proportion of methylated DNA at a specific CpG site within a sample) and Tensor S×C×T (a multi-dimensional array).

RNA methylation 2022, comprising m5C (s a known RNA modifications), m6A markers (are distinct types of RNA methylation that play crucial roles in gene expression and regulation), expression levels (referrer to the amount of gene product (usually RNA or protein) produced by a gene), UMI-corrected (data that has been processed to account for errors in sequencing and amplification by leveraging Unique Molecular Identifiers (UMIs). UMIs are unique DNA or RNA tags added to each molecule before amplification, allowing computational methods to identify and correct artifacts like PCR-induced errors (substitutions, insertions, or deletions).

Proteomics 2023, MS intensity (refers to the strength of the signal detected, representing the relative abundance of ions at a specific mass-to-charge ratio (m/z)), TMM-scaled (refers to data that has been normalized using the Trimmed Mean of M-values (TMM) normalization method, a common technique in RNA-Seq analysis to adjust for differences in library sizes and other technical biases between samples. The TMM method calculates a scaling factor for each sample by taking a weighted, trimmed mean of the log-fold changes (M-values) and absolute expression levels (A-values) between the sample and a reference, then uses these factors to make the samples comparable for downstream analyses like differential expression testing), 6,200 proteins (measuring over 6,200 proteins in a sample using Data-independent acquisition mass spectrometry DIA-MS)

Layer 3 passes information to the Tensor Fusion 2025 (refers to the process of combining multiple tensors, often representing different modalities or features, into a single tensor to create a more comprehensive representation for further processing) which creates a Unified Molecular signature 2020 (which is a consistent molecular pattern (like gene expression) found across different patient populations or studies that can accurately predict a disease state or risk)

The system provides:
a. Clinical Diagnostics: Immediate normalization allows immediate analysis of patient samples, enabling rapid diagnostic decisions in acute leukemia cases where treatment delays impact outcomes.
b. Multi-Center Trials: Cross-platform harmonization enables aggregation of data from multiple clinical sites using different sequencing technologies, increasing statistical power and generalizability.
c. Precision Medicine: Accurate normalization is essential for matching patient expression profiles to treatment response databases, enabling personalized therapy selection.
d. Drug Development: Normalized expression data facilitates biomarker discovery and validation across diverse patient populations and experimental conditions.
e. Quality Assurance: The system serves as a quality control checkpoint, identifying problematic samples before expensive downstream analyses.
f. Algorithm Development: AI-ready outputs enable rapid development and validation of new diagnostic algorithms without preprocessing bottlenecks.
g. In clinical pilot deployments, the system enabled oncologists to review normalized data during the same patient encounter.

While the preferred embodiment processes RNA, protein, and genomic data, alternative embodiments may include:
a. Single-modality versions optimized for specific data types
b. Edge computing implementations for point-of-care devices
c. Federated learning versions that improve without centralizing patient data
d. Specialized versions for other cancers beyond leukemia
e. Integration with specific electronic health record systems
f. Versions optimized for research versus clinical applications

These variations fall within the scope of the core invention while addressing specific use cases or deployment scenarios.

The differences between existing known methods and the disclosed method can be summarized as:

The present invention differs fundamentally from existing batch correction and multi-omics integration methods in both architecture and performance.

Unlike tools such as Seurat, Harmony, MOFA+, and related algorithms—which operate on pre-normalized or single-modality datasets and typically require offline computation. The disclosed system executes real-time (<3 seconds per sample) multi-modal tensor decomposition directly on raw counts from heterogeneous assays (RNA expression, proteomics, variant data).

In addition, the invention uniquely integrates dynamic atlas matching based on demographic and platform metadata (age±5 years, sex match, same platform, minimum N≥100 controls) and achieves defined quantitative thresholds of ≥94% batch variance reduction and 98% biological variance retention. No known prior art system combines these elements into a single clinical-grade workflow suitable for diagnostic deployment.

Table 1 below compares representative prior art systems to the present invention, highlighting that no single reference teaches or suggests the claimed combination of features.

TABLE 1

| | | Dynamic Atlas | | Temporal | |
|---|---|---|---|---|---|
| | | Matching | Cross-Platform | Methylation/ | Quantum-Inspired |
| Tool/Method | Primary Scope | (age/sex/platform) | Concordance | Early Detection | Optimization |
| [symbol] | Single cell multi- [symbol] | No | Yes, not clinical, | No | No |
| WNN | integration (RNA-ATAC) | | grade | | |
| Harmony | Batch correction for | No | Yes, not clinical, | No | No |
| | scRNA-seq | | grade | | |
| MOFA/ | Multi- [symbol] factor | No | Some (via factors) | No | No |
| MOFA+ | analysis (bulk & single- | | | | |
| | cell) | | | | |
| [symbol] | Pancrama stitching for | No | Yes, scRNA only | No | No |
| | scRNA | | | | |
| ComBat( [symbol] ) | Batch correction for | No | Some, not multi- | No | No |
| | bulk data | | omics | | |
| [symbol] | Batch correction (bulk) | No | Limited | No | No |
| MNN Correct | Single-cell batch | No | Yes, scRNA only | No | No |
| ( [symbol] ) | correction via [symbol] | | | | |
| | [symbol] | | | | |
| [symbol] | Integrat [symbol] NMF for | No | Yes (through | No | No |
| | multi- [symbol] | | factors) | | |
| [symbol] | Batch balanced kNN | No | Yes, scRNA only | No | No |
| | for scRNA integration | | | | |
| | ( [symbol] ) | | | | |
| [symbol] | Deep generative | No | Yes (latent space) | No | No |
| MultiVI | models for multi-omics | | | | |

[symbol]

indicates data missing or illegible when filed

### EXAMPLES

The following examples provide pseudocode, formulas, and worked examples supporting the embodiments of the claimed invention.

### Example 1—Pseudocode: End-to-End Multi-Modal Normalization

```
: End-to-End Normalization (RNA+Proteomics+Variants).
Hardware: dual 24-core CPUs; 256 GB RAM; optional A100 40 GB. Data: N=480;
RNA (40M reads/sample), proteomics (6,200 proteins), variants (22k/sample).
Procedure: ingest → UMI/TMM → proteomics normalize/log/impute K=3 → variant
summarization → build T → randomized SVD (power=2, oversample=32) → auto-
rank → project/reconstruct → atlas match → outputs + QC. Observed: ~2.5
s/sample (GPU), ~4.9 s (CPU); PVCA batch ~23%→~6%; bio variance ≥98%; cross-
platform r² ~0.95-0.97.
Inputs: RNA counts X_rna (S×G), Proteomics X_prot (S×P), Variant features X_var
(S×V), batch labels, clinical covariates, and reference atlas A.
Per-modality preprocessing: UMI de-duplication, TMM scaling for RNA, size-factor
scaling for proteomics.
Tensor batch effect removal: Build tensor (S×F×M), unfold mode-1, truncated SVD,
select rank r, remove batch components, refold.
Unified normalization equation per feature f, sample s:
X_norm(s,f) = α_m * UMI_correct(s,f) + β_m * TMM_scale(s,f) + γ_m *
Batch_remove(s,f)
Weights α_m, β_m, γ_m learned to minimize batch variance (PVCA) while
preserving ≥98% biological variance.
Dynamic atlas matching: select control cohort with |age_s − age_c| ≤ 5, same sex,
same platform, N≥100; compute per-feature Z-scores.
procedure NORMALIZE_MULTI_MODAL(X_rna, X_prot, X_var, meta, atlas):
  X_rna <− UMI_DEDUP(X_rna)
  X_rna <− TMM_SCALE(X_rna)
  X_tensor <− STACK_MODALITIES(X_rna, X_prot, X_var)
  X1 <− MODE1_UNFOLD(X_tensor)
  U, S, Vt <− TRUNCATED_SVD(X1)
  r <− AUTO_RANK(S, X1)
  B <− U[:,0:r]
  mu <− FEATURE_MEAN(X1)
  X1_corr <− X1 − B @ (B.T @ X1) + (mu @ ONES_ROW(S).T)
  Xcorr_tensor <− MODE1_REFOLD(X1_corr, S, F, M)
  alpha, beta, gamma <− LEARN_WEIGHTS(...)
  Xnorm <− COMBINE(alpha, beta, gamma, UMI_CORRECTED, TMM_SCALED,
Xcorr_tensor)
  C <− SELECT_ATLAS_COHORT(atlas, meta.age, meta.sex, meta.platform)
  mu_f, sigma_f <− FEATURE_STATS(C)
  Z <− (Xnorm − mu_f) / sigma_f
  return Xnorm, Z, {r, alpha, beta, gamma}
end
procedure NORMALIZE_MULTI_MODAL(X_rna, X_prot, X_var, meta, atlas):
    X_rna <− UMI_DEDUP(X_rna)
    X_rna <− TMM_SCALE(X_rna)
    X_tensor <− STACK_MODALITIES(X_rna, X_prot, X_var)
    X1 <− MODE1_UNFOLD(X_tensor)
    U, S, Vt <− TRUNCATED_SVD(X1)
    r <− AUTO_RANK(S, X1)
    B <− U[:,0:r]
    mu <− FEATURE_MEAN(X1)
    X1_corr <− X1 − B @ (B.T @ X1) + (mu @ ONES_ROW(S).T)
    Xcorr_tensor <− MODE1_REFOLD(X1_corr, S, F, M)
    alpha, beta, gamma <− LEARN_WEIGHTS(...)
    Xnorm <− COMBINE(alpha, beta, gamma, UMI_CORRECTED, TMM_SCALED,
Xcorr_tensor)
    C <− SELECT_ATLAS_COHORT(atlas, meta.age, meta.sex, meta.platform)
    mu_f, sigma_f <− FEATURE_STATS(C)
    Z <− (Xnorm − mu_f) / sigma_f
    return Xnorm, Z, {r, alpha, beta, gamma}
```

### Example 2—Worked Numeric Toy Example

Latency Under Load. N=2,400; P95≤3.0 s (GPU), ≤6.2 s (CPU).
Four samples, four genes, two batches. Show batch shift removal using TMM scaling and rank-1 SVD batch correction. Provide numeric Table 2 and resulting corrected matrix.

TABLE 2

| | Input Data/ | Controls/ | Expected |
|---|---|---|---|
| STEP | Parameters | Quality Checks | Output |
| Data Ingestion | RNA-seq FASTQ/BAM, | File format validation, | |
| | Proteomics mzML/mzXML, | Metadata | Validated raw |
| | Genomic VCF; Clinical | completeness check | data streams |
| | metadata | | |
| Modality- | UMI deduplication, TMM | Coverage uniformity, | Preprocessed |
| Specific | scaling, Platform- specific | Outlier detection | data with |
| Preprocessing | adjustments | | reduced |
| | | | technical |
| | | | bias |
| Multi-Modal | Parallel pipelines for RNA, | Cross-platform | Harmonized |
| Harmonization | protein, variant data; Cross- | concordance ≥0.95 | multi-modal |
| | modal scaling factors | | dataset |
| Tensor-Based | Tensor decomposition | Batch variance | Batch- |
| Batch Effect | rank = r | reduction ≥94%, | corrected |
| Correction | (auto); μ for global mean | Biological variance | dataset |
| | preservation | retention ≥98% | preserving |
| | | | true biology |
| Reference | Reference cohort ≥100 | Diverse, sex-balanced, | Matched |
| Atlas Matching | samples; Age ±5 years, Sex | platform-specific atlas | normalized |
| | exact, Platform matched | | dataset to |
| | | | healthy |
| | | | baseline |
| Output | Generate HDF5, FHIR JSON, | QC metrics panel | Standardized, |
| Formatting | Tab- delimited; Include QC | review, Reproducibility | reproducible |
| | metrics | logs | data |
| | | | structures |
| Integration with | API-ready formats, Risk | HIPAA/GDPR | Integrated |
| AI Diagnostic | scores, Predictive analytics | compliance, | clinical |
| Platform | | Latency <3 | decision |
| | | sec/sample | support |
| | | | outputs |

Date Ingestion (RNA-sequence, Proteomics, Genomics),
Modality-specific Preprocessing (UMI correction, TMM Scaling))
Multi-Modal Harmonization engine,
Tensor-based batch effect correction,
Reference Atlas Matching (Age/Sex/Platform matched),
Output formatting (FHIR, JSON, HDFS, Tabular, and then Integration with AI Diagnostics Platform.

### Example 3—Real-World Style Evaluation

Cross-Platform Harmonization. Replicates Illumina/ONT+2 MS instruments; RNA r²≥0.95; proteomics r²≥0.93; pre-corr r²≤0.80.

Dataset: 1,200 leukemia cases+3,600 controls, three modalities. Hardware: GPU+CPU. Steps: preprocessing, tensor batch correction, weight learning, atlas matching.

### Metrics:

Runtime <3s/sample
PVCA batch variance 56%
Biological variance retention 98%
Cross-platform r²≥0.95

### Example 4—Atlas Matching Algorithm—Batch Removal & Signal Retention

PVCA batch ≤6%; subtype AUC +0.09; control variance ≥98%.

```
procedure SELECT_ATLAS_COHORT (atlas, age, sex, platform, Nmin=100,
tol_age=5):
  C <− FILTER(atlas, |age_c − age| <= tol_age AND sex_c == sex AND
PLATFORM_COMPAT(platform_c, platform))
  if |C| < Nmin:
      C <− FILTER(atlas, sex_c == sex AND PLATFORM_FAMILY(platform_c) ==
PLATFORM_FAMILY(platform))
      while |C| < Nmin and tol_age <= 15:
        tol_age <− tol_age + 2
        C <− FILTER(atlas, |age_c − age| <= tol_age AND sex_c == sex)
  return RANDOM_SAMPLE(C, Nmin) if |C| >= Nmin else C
end
procedure SELECT_ATLAS_COHORT(atlas, age, sex, platform, Nmin=100,
tol_age=5):
    C <− FILTER(atlas, |age_c − age| <= tol_age AND sex_c == sex AND
PLATFORM_COMPAT(platform_c, platform))
    if |C| < Nmin:
        C <− FILTER(atlas, sex_c == sex AND PLATFORM_FAMILY(platform_c) ==
PLATFORM_FAMILY(platform))
        while |C| < Nmin and tol_age <= 15:
          tol_age <− tol_age + 2
          C <− FILTER(atlas, |age_c − age| <= tol_age AND sex_c == sex)
    return RANDOM_SAMPLE(C, Nmin) if |C| >= Nmin else C
end
```

TABLE 3
Table 3 - Mini Dataset for Atlas Matching Example

| | | | | | | RNA | RNA | | |
|---|---|---|---|---|---|---|---|---|---|
| Patient | | | Tissue | GENE- | RNA | Methylation | Methylation | LAB | Lab |
| ID | AGE | SEX | Type | RNA | Expression | Type | Conf | Hb | WBC |
| NH-GEN- | 61 | M | liver | BRCA1 | 6.34 | m5C | 0.97 | 14.4 | 5.7 |
| 000001 | | | | | | | | | |
| NH-GEN- | 69 | M | liver | METTL3 | 2.46 | m5C | 0.85 | 14.9 | 9.9 |
| 000003 | | | | | | | | | |
| NH-GEN- | 58 | M | liver | TP53 | 4.87 | m1A | 0.91 | 13.2 | 6.5 |
| 000010 | | | | | | | | | |
| NH-GEN- | 63 | M | liver | KRAS | 5.41 | m5C | 0.94 | 14.1 | 7.4 |
| 000015 | | | | | | | | | |
| NH-GEN- | 60 | M | liver | BRCA2 | 3.76 | m1A | 0.88 | 12.9 | 6.8 |
| 000018 | | | | | | | | | |
| NH-GEN- | 65 | M | liver | METTL14 | 2.89 | m5C | 0.9 | 14 | |
| 000022 | | | | | | | | | |

### Example 5—Implementation Notes

Atlas Ablation. Dynamic atlas lowers PVCA by 4-6pp; Δr²+0.03.
Use randomized truncated SVD for speed; center and scale features; clip extreme Z-scores; default parameters: τ=0.9, λ=1e-3, N_min=100. Outputs in HDF5, FHIR JSON, TSV.
The steps are shown in Table 4 below.

TABLE 4

| Step | Input Data/Parameters | Controls/Quality Checks | Expected Output |
|---|---|---|---|
| Sample Acquisition | cfDNA from plasma, RNA from blood, | Sample handling SOP compliance, | High-quality biospecimens |
| | Proteomics datasets | duplicate sample detection | with metadata |
| Initial Quality | Concentration, purity metrics, | Integrity thresholds, contamination | Validates raw data streams |
| Control | metadata completeness | screening | |
| Data | Sequence alignment, modality-specific | Coverage uniformity, outlier removal, | Clean, normalized datasets |
| Preprocessing | normalization, error correction | technical variance assessment | |
| Feature | Detection of CpG methylation patterns, | Feature selection reproducibility, | Structured multi-modal |
| Extraction | SNVs/indels, proteomic intensity peaks | cross-modality concordance | feature set |
| Model | Machine learning diagnostic models, risk | Model performance (AUC, sensitivity, | Diagnostic predictions, risk |
| Application | scoring algorithms | specificity) | scores |
| Validation & Cross- | Reference atlas lookup, control cohort | Concordance with reference baselines, | Validation metrics, matched |
| Reference | comparison, cross-validation | inter-run reproducibility | baselines |
| Output & Clinical | Generate clinical report, API, ready | Data security checks, format | Actionable clinical output |
| Integration | data, EHR integration files | validation, regulatory compliance | and system integration |

Sample Acquisition (cfDNA,RNA, Proteomics),
Initial quality control (Integrity, purity, metadata check),
Data Processing (Alignment, Normalization, error correction)
Feature extraction (Methylation signatures, variant, proteomic markers),
Model application (diagnostics algorithm, risk scoring),
Validation and cross reference(reference atlas, control cohorts), and
Output and clinical integration (reports, API feeds, HER integration).

### Example 6—PVCA-Validated Multi-Modal Normalization

Objective. Demonstrate that the claimed system (i) achieves <3 s/sample end-to-end normalization, (ii) reduces batch variance by ≥94% (PVCA), and (iii) retains 98% biological variance, on RNA+proteomics (and optionally variant) data.

### Inputs.

RNA counts matrix X_rna (S×G) with UMI barcodes; proteomics intensity matrix X_prot (S×P); optional variant features X_var (S×V); batch labels; age/sex/platform metadata; leukemia reference atlas. Support: modalities+atlas.

### Methods.

### Per-Modality Preprocessing.

UMI_DEDUP(X_rna); TMM_SCALE(X_rna); size-factor scaling for X_prot; optionally depth-aware scaling for X_var. Support: preprocessing steps.

### Tensor Batch Removal.

STACK_MODALITIES→MODE1_UNFOLD→TRUNCATED_SVD; AUTO_RANK selects r; compute X1_corr=X1−B(B^{T}X1)+μ; refold to tensor.

### Normalization Combiner.

Compute X_norm(s,f)=Σ_m[α_m·UMI_correct+β_m·TMM_scale+γ_m·Batch_remove]; learn {α,β,γ} to minimize PVCA batch variance with a retention constraint.

### Reference-Atlas Z-Scoring.

SELECT_ATLAS_COHORT(age±5, sex, platform, N≥100); compute μ_f, α_f on cohort; compute Z=(X_norm−μ_f)/σ_f.

### Metrics.

Compute PVCA on batch factor pre and post normalization; compute fraction of variance explained by biological labels pre/post. Record wall-clock normalization time per sample on the stated hardware.

TABLE 5

| | | | | | | | | Runtime | Hardware | | |
|---|---|---|---|---|---|---|---|---|---|---|---|
| | | | | | | Batch | Biological | per | [symbol] | | |
| Modality | Dataset name/ | | | PVCA batch | PVCA batch | variance | variance | sample | (CPU/GPU/ | | |
| [symbol] | cohort ID | N samples | Batches | variance (pre) | variance (post) | [symbol] | [symbol] | [symbol] | RAM) | [symbol] | Notes |
| RNA | [symbol] | [symbol] | 4 | 0.01 | 0.00 | 100.00 | [symbol] | [symbol] | [symbol] | — | [symbol] |
| [symbol] | [symbol] | [symbol] | 4 | 0.02 | 0.00 | 100.00 | [symbol] | [symbol] | [symbol] | — | [symbol] |

[symbol]

indicates data missing or illegible when filed

In some embodiments the method or methods described above may be executed or carried out by a computing system including a tangible computer-readable storage medium, also described herein as a storage machine, that holds machine-readable instructions executable by a logic machine (i.e. a processor or programmable control device) to provide, implement, perform, and/or enact the above described methods, processes and/or tasks. When such methods and processes are implemented, the state of the storage machine may be changed to hold different data. For example, the storage machine may include memory devices such as various hard disk drives, CD, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI) or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, keyboard or gaming controller. For example, a user input may indicate a request that certain task is to be executed by the computing system, such as requesting the computing system to display any of the above-described information, or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API).

One issue with any online system that interfaces with government, health care, patient health data or large business systems is security. There is a need to ensure that the individual, smart device or computer system interacting with the system is a sanctioned user or sanctioned device. The biggest issues in cyber security are:
Configuration Mistakes.
Poor Cyber Hygiene.
Cloud Vulnerabilities.
Mobile Device Vulnerabilities.
Internet of Things.
Ransomware.
Poor Data Management.
Inadequate Post-Attack Procedures.

The instant invention addresses the problem of Cloud Vulnerabilities and Mobile Device Vulnerabilities. By creating a system that allows access to the systems while ensuring that the devices interacting with the system are sanctioned and allowed to use the system. The instant invention uses a user name, password, and a virtual private network (VPN) if desired and then overlays a second security measure comprising at least one of the following two methods which can be employed to solve the authorized connection problem which is at the heart of any Cloud Vulnerabilities and Mobile Device Vulnerabilities issues. The system can use a Global Positioning System (GPS) location filter or an Internet Protocol (IP) address (IP address) filter that allows only those devices that either are from the correct or allowed GPS locations contained in a sanction GPS locations file on the cloud server or have the correct IP address contained in a sanction IP address file on the cloud server. The instant invention can use these filters either individually or in combination to limit access to the system. Because of these security measures used in conjunction and integrated into the system of the instant invention the result is a system that can only be implemented with a dedicated network of computerized devices. That network comprises of a cloud server and remote sanctioned smart devices that have either a validated IP address or are located in a sanctioned location that is verified by the GPS location.

Alternatively, the system can be configured to allow only single purpose devices configured such that they can only access the system and the subsequent remote of cloud server. These single purpose devices have been optimized to provide only access to the system and ca also have the additional security protocols of a sanctioned GPS location or sanction internet protocol address.

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments; it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

We claim:

1. A computer-implemented system for normalizing multi-modal molecular expression data in leukemia diagnostics, comprising:
a data ingestion module configured to simultaneously receive RNA sequencing data, proteomic data, and genomic variant data from multiple platforms;
a multi-modal harmonization engine applying parallel processing pipelines optimized for each data modality;
a batch effect correction module utilizing tensor decomposition to remove technical artifacts while preserving biological signals;
a reference atlas containing normalized expression profiles from healthy control samples; and
an output formatting engine generating standardized data structures compatible with automated clinical decision support systems, wherein the system rapidly processes multi-modal data per sample.

2. The system of claim 1, wherein the multi-modal harmonization engine implements unique molecular identifier-based correction, trimmed mean normalization, and modality-specific scaling factors.

3. The system of claim 1, wherein the batch effect correction module achieves a reduction in batch-associated variance.

4. The system of claim 1, wherein the reference atlas contains normalized expression profiles of healthy control samples.

5. The system of claim 4, wherein the reference atlas includes age-stratified cohorts spanning pediatric to geriatric populations, sex-balanced representation, and ethnically diverse samples.

6. The system of claim 1, further comprising a methylation mapping module that performs temporal analysis of epigenetic changes.

7. The system of claim 6, wherein the methylation mapping module generates Methylation Kinetic Codes (MK-Codes) capturing temporal dynamics of methylation changes across analyzed loci.

8. A method for normalizing multi-modal molecular data in leukemia diagnostics, comprising:
receiving heterogeneous molecular data streams including RNA expression, protein abundance, and genomic variants;
applying modality-specific preprocessing including unique molecular identifier correction and platform-specific adjustments;
performing tensor-based batch effect correction that preserves biological variation;
dynamically selecting reference cohorts from a curated atlas based on sample characteristics; and
generating normalized expression matrices compatible with downstream analysis platforms, wherein all steps are completed rapidly per sample.

9. The method of claim 8, wherein the tensor-based batch effect correction removes technical artifacts while retaining biological signal variance.

10. The method of claim 8, wherein dynamically selecting reference cohorts comprises matching age, exact sex, and compatible sequencing platform.

11. The method of claim 8, further comprising performing quality control steps at each processing stage with sub-minute processing metric reporting.

12. The method of claim 8, wherein the normalized expression matrices are directly compatible with artificial intelligence diagnostic systems without additional preprocessing.

13. The method of claim 12, further comprising generating Methylation Kinetic Codes (MK-Codes) capturing temporal dynamics of methylation changes across analyzed loci.

14. The method of claim 13, wherein the MK-Codes enable prediction of leukemic transformation before conventional diagnostic markers become detectable.

15. A non-transitory computer-readable medium storing instructions that, when executed by a processor, cause the processor to perform a method for normalizing multi-modal molecular data in leukemia diagnostics, the method comprising:
ingesting RNA sequencing data, proteomic data, and genomic variant data from multiple platforms;
harmonizing the ingested data using parallel processing pipelines optimized for each data modality;
correcting batch effects using tensor decomposition while preserving biological signals;
comparing the corrected data against a reference atlas of healthy control samples; and
outputting standardized data structures compatible with artificial intelligence diagnostic systems, wherein the method is performed in rapidly per sample.

16. The non-transitory computer-readable medium of claim 15, wherein harmonizing the ingested data comprises applying unique molecular identifier-based correction, trimmed mean normalization, and modality-specific scaling factors.

17. The non-transitory computer-readable medium of claim 15, wherein correcting batch effects achieves a reduction in batch-associated variance while retaining biological signal variance.

18. The non-transitory computer-readable medium of claim 15, wherein the reference atlas contains normalized expression profiles from healthy control samples spanning pediatric to geriatric populations.

19. The non-transitory computer-readable medium of claim 18, wherein comparing the corrected data against the reference atlas comprises dynamically selecting reference cohorts based on matching age, exact sex, and compatible sequencing platform.

20. The non-transitory computer-readable medium of claim 19, wherein the method further comprises generating Methylation Kinetic Codes (MK-Codes) capturing temporal dynamics of methylation changes across analyzed loci, wherein the MK-Codes enable prediction of leukemic transformation.

21. The system of claim 1, further comprising a quantum-inspired optimization engine that: applies quadratic unconstrained binary optimization (QUBO) modeling to methylation state transitions; implements simulated annealing with adaptive temperature scheduling; utilizes tensor network contractions for high-dimensional pattern recognition; and identifies optimal methylation pathways through energy minimization algorithms.

22. The system of claim 21, wherein the quantum-inspired optimization engine employs Matrix Product State decomposition to model methylation correlations across genomic regions.

23. The system of claim 21, wherein the QUBO formulation assigns cost functions based on biological plausibility, population frequency data, and association with leukemic outcomes.
